(19) United States
(12) Patent Application Publication
(10) Pub. No.: US 2025/0308880 A1
(43) Pub. Date: Oct. 2, 2025

(54) FAR ULTRAVIOLET LIGHT EMITTING DEVICE

(71) Applicant: LG ELECTRONICS INC., Seoul (KR)

(72) Inventors: Tae Young KIM, Seoul (KR); Muntae JUNG, Seoul (KR)

(21) Appl. No.: 19/090,953

(22) Filed: Mar. 26, 2025

(30) Foreign Application Priority Data
Mar. 29, 2024 (WO) PCT/KR2024/004054
Dec. 4, 2024 (KR) 10-2024-0178752

(51) Int. Cl. H01J 61/10; A61L 2/10; A61L 9/20; H01J 61/12; H01J 61/40

(52) U.S. Cl. CPC H01J 61/10; A61L 2/10; A61L 9/20; H01J 61/12; H01J 61/40; A61L 2202/11; A61L 2202/25; A61L 2209/12

(57) ABSTRACT

The present disclosure relates to a far ultraviolet light emitting device and a home appliance having the same. The present disclosure may include a first electrode part 130A and a second electrode part 130B spaced apart from the first electrode part 130A. A barrier may be arranged between the first electrode part 130A and the second electrode part 130B. An ultraviolet lamp 140 may have an electrode connection surface 141B arranged toward the surface of the first electrode part 130A, the surface of the second electrode part 130B, and the surface of the barrier 126, and a light extraction surface 141A arranged toward the opposite side of the electrode connection surface 141B. In this case, at least one of the surfaces of the first electrode part 130A or second electrode part 130B and the surface of the barrier 126 may be spaced apart from each other to form a gap part 136. The gap part 136 may be opened toward the electrode connection surface 141B.

### CROSS-REFERENCE TO RELATED PATENT APPLICATIONS

The present application claims priority under 35 U.S.C. 119 to PCT/KR2024/004054, filed Mar. 29, 2024 and Korean Patent Application No. 10-2024-0178752, filed Dec. 4, 2024, whose entire disclosures are hereby incorporated by reference.

### TECHNICAL FIELD

The present disclosure relates to a far ultraviolet light emitting device.

### BACKGROUND ART

Ultraviolet lamps generate ultraviolet light and are used in various fields for the purpose of sterilization of bacteria and fungi. The ultraviolet lamp generates ultraviolet rays (UV) of various wavelengths by the material provided inside the lamp. For example, the ultraviolet lamp may generate UV-A (315 nm to 400 nm), UV-B (280 nm to 315 nm), UV-C (100 nm to 280 nm), or the like.

Among them, ultraviolet rays with a wavelength corresponding to UV-C are the most excellent in sterilization. When UV-C is irradiated to bacteria and fungi, DNA of bacteria and fungi is damaged and killed. That is, UV-C has an effective sterilizing power against various bacteria by damaging the DNA of an organism. Therefore, sterilizing with ultraviolet lamps is more efficient than sterilizing with heat, chemicals, ozone, or radiation, and the like.

Recently, devices are being developed that selectively irradiate ultraviolet rays in the wavelength range of 200 nm to 230 nm, which are known to have a sterilizing effect while being harmless to the human body, especially the 222 nm wavelength known as Far UVC. To obtain light with a wavelength of 222 nm, a dust-proof gas is mainly used as KrCl, an excimer gas.

Such an ultraviolet lamp creates a vacuum atmosphere in the inner space of the tube and requires gas to be injected between them. The inner space becomes a discharge space and may generate ultraviolet rays through light emission. The ultraviolet lamp induces discharge by applying a voltage through an electrode.

Technologies with ultraviolet lamps in the form of circular and rectangular tubes are disclosed in Korean Patent Application Publication No. 10-2021-0049890 (prior art 1) and Korean Registered Patent Publication No. 10-1646862 (prior art 2), respectively. Prior art 1 and prior art 2 each include technologies to discharge the ultraviolet lamp by contacting an electrode with the ultraviolet lamp and applying a voltage to the ultraviolet lamp through the electrode.

However, conventional ultraviolet lamps, including prior arts 1 and 2, are often not operated because they are sensitive to the installation environment, that is, temperature, humidity or atmospheric pressure. For example, when it is in a low temperature state or when it is used after not being used for a long time, the ultraviolet lamp may not start, or it may take a long time to start. When the ultraviolet lamp is not started, stable discharge of the ultraviolet lamp may also not be achieved.

In order to solve these problems, a technology that provides an auxiliary means for starting an ultraviolet lamp is known. For example, in the International Publication Patent WO 2021/025064 (prior art 3), a technology that helps the initial driving of an ultraviolet lamp by providing an auxiliary starting light source is disclosed. In addition, Korean Publication Patent 10-2023-0163552 (prior art 4) discloses a technology that helps the initial driving of an ultraviolet lamp by providing a conductor as a separate auxiliary electrode.

However, since prior arts 3 and 4 require additional electrodes or light sources in addition to electrodes that apply voltage to the ultraviolet lamp, the overall structure of the ultraviolet generator is complicated and its size increases, and the number of parts increases, thereby increasing the manufacturing cost. In addition, since auxiliary electrodes or auxiliary light sources must be properly controlled, a structure for controlling them must be added, and the overall size of the ultraviolet generator also increases.

Alternatively, a high voltage may be applied to the ultraviolet lamp. However, in order to apply a high voltage, there is a problem that the power supply for lighting becomes larger. In addition, sparks are generated when a high voltage is applied, causing problems that reduce the stability of the ultraviolet generator.

### Documents of Related Art

(Prior Art 1) Korean Patent Application No. 10-2021-0049890
(Prior Art 2) Korean Registered Patent No. 10-1646862
(Prior Art 3) International Patent Application WO 2021/025064
(Prior Art 4) Korean Patent Application No. 10-2023-0163552

### DETAILED DESCRIPTION OF THE DISCLOSURE

### Technical Problem

The present disclosure is intended to solve the problems of the prior art as described above, and the purpose of the present disclosure is to ensure that the initial drive of an ultraviolet lamp is stably performed without a separate auxiliary lighting device in an ultraviolet generator.

Another purpose of the present disclosure is to prevent sparks from being generated by a voltage applied during the initial drive of the ultraviolet generator.

Still another purpose of the present disclosure is to reduce the number of parts and simplify the structure of the ultraviolet generator.

Still another purpose of the present disclosure is to configure each of a light extraction surface and an electrode connection surface of the ultraviolet generator as a flat surface.

### Problem Solving

According to the features of the present disclosure for achieving the above-mentioned purposes, the present disclosure may include a first electrode part having a first electrode surface and a second electrode part disposed to be spaced apart from the first electrode part and having a second electrode surface facing the first electrode surface.

A barrier may be arranged between the first electrode surface and the second electrode surface. An ultraviolet lamp may have an electrode connection surface arranged toward a surface of the first electrode part, a surface of the second electrode part, and a surface of the barrier, and a light extraction surface arranged toward the opposite side of the electrode connection surface.

In this case, a stepped surface forming a stepped portion may be included in either the first electrode surface or the second electrode surface. A surface of the barrier may be spaced apart from the stepped surface. The barrier may be a kind of dielectric arranged between the two electrode parts, and may induce lighting of the ultraviolet lamp through electric polarization.

The electrode connection surface may face an upper surface of the first electrode part, an upper surface of the barrier, and an upper surface of the second electrode part. Such an arrangement may prevent sparks from occurring when a voltage is applied to the two electrode parts.

In addition, the first electrode surface may include a barrier facing surface facing the surface of the barrier and spaced apart from the surface of the barrier by a first distance. The first electrode surface may include the stepped surface facing the surface of the barrier and spaced apart from the surface of the barrier by a second distance longer than the first distance. The first electrode surface may include a stepped connection surface connecting the barrier facing surface and the stepped surface.

In addition, the upper surface of the barrier facing the electrode connection surface may be spaced apart from the electrode connection surface.

In addition, the upper surface of the barrier may be spaced apart from the electrode connection surface. A distance between the upper surface of the barrier and the electrode connection surface may be shorter than a distance (D1A) between the stepped surface and the side surface of the barrier.

In addition, a height at which the surface of the barrier facing the stepped surface protrudes from the stepped connection surface toward the electrode connection surface is smaller than the sum (D1A+D2A) of the distance (D1A) between the stepped surface and the surface of the barrier and a distance (D2A) between the stepped connection surface and the electrode connection surface. In this way, the irradiation rate of the ultraviolet lamp may be increased while effectively preventing sparking.

A thickness of the barrier (D3) based on the direction in which the first electrode part and the second electrode part are spaced from each other may be smaller than the sum (D1A+D2A) of the distance (D1A) between the stepped surface and the surface of the barrier and the distance (D2A) between the stepped connection surface and the electrode connection surface.

The stepped surface is arranged closer to the electrode connection surface than the barrier-facing surface.

In addition, the stepped connection surface may be spaced apart from the electrode connection surface, while facing the electrode connection surface.

In addition, the stepped surface may be formed in a shape in which a portion of the first electrode surface is recessed in a direction away from the surface of the barrier from the barrier facing surface.

In addition, the distance (D1A) between the stepped surface and the surface of the barrier may be 0.8 to 1.2 times the distance (D2A) between the stepped connection surface and the electrode connection surface.

And, the distance (DIA) between the stepped surface and the surface of the barrier may be greater than or equal to the thickness of the barrier (D3) based on the direction in which the first electrode part and the second electrode part are spaced from each other.

In addition, a gap part may be formed by being surrounded by the stepped surface, the surface of the barrier, and the electrode connection surface.

And, at least one of the electrode connection surface or the light extraction surface is a plane shape.

In addition, the barrier is provided with a barrier protrusion protruding toward a surface of the first electrode part and a surface of the second electrode part. In addition, electrode grooves in which the barrier protrusions are disposed are formed in the first electrode part and the second electrode part, respectively.

The stepped surface may be formed continuously along a direction orthogonal to the direction in which the stepped surface and the surface of the barrier are separated from each other.

The stepped surface may have an auxiliary electrode protruding toward the surface of the barrier.

The surface of the ultraviolet lamp is curved, and curved mounting grooves in which the surface of the ultraviolet lamp is mounted are formed in the first electrode part and the second electrode part.

In addition, a gap part recessed in a direction spaced apart from the electrode connection surface and the surface of the barrier, respectively, may be formed in the first electrode part or the second electrode part.

In addition, the present disclosure may include a first electrode part and a second electrode part spaced apart from the first electrode part. A barrier may be arranged between the first electrode part and the second electrode part. An ultraviolet lamp may have an electrode connection surface arranged toward the surface of the first electrode part, the surface of the second electrode part, and the surface of the barrier, and a light extraction surface arranged toward the opposite side of the electrode connection surface. In this case, the first electrode part and the surface of the barrier, and the second electrode part and the surface of the barrier may be spaced apart from each other to form a plurality of gap parts that open toward the electrode connection surface.

Meanwhile, the gap part that opens toward the electrode connection surface is formed between the first electrode part and the second electrode part, and the barrier is disposed between the first electrode part and the second electrode part to divide the gap part into a first gap part and a second gap part.

In addition, at least two different surfaces of the gap part may be formed by the first electrode part or the second electrode part, respectively.

In addition, the surface of the first electrode part and the surface of the barrier are spaced apart to form a gap part that opens toward the electrode connection surface. In the first electrode part, a stepped surface having a height lower than that of other parts toward the electrode connection surface may be formed, and the gap part may be formed between the stepped surface and the electrode connection surface.

In addition, the surface of the first electrode part and the surface of the barrier are spaced apart to form a gap part that opens toward the electrode connection surface. A part of the first electrode part may be recessed in a direction away from the surface of the barrier to form the electrode part.

### Effects of the Disclosure

Afar ultraviolet light emitting device of the present disclosure as described above have the following effects.

In the present disclosure, a barrier is arranged between two electrode parts, and an ultraviolet lamp may be arranged to cross the two electrode parts and the barrier. In this case, the barrier may be a kind of dielectric arranged between the two electrode parts, and may induce the lighting of the ultraviolet lamp through electric polarization. In this way, the present disclosure has the effect of lighting the ultraviolet lamp without a separate auxiliary lighting device, and stably performing the initial drive of a far ultraviolet light emitting device.

In addition, a gap part may be formed between the barrier and the two electrode parts in the present disclosure. The gap part may prevent sparks from occurring when voltage is applied to the two electrode parts. Therefore, even when a high voltage is applied to the ultraviolet lamp, sparks are prevented, and thus the stability of the far ultraviolet light emitting device may be improved.

In addition, the barrier may be integrally provided with a lamp housing of the far ultraviolet light emitting device in the present disclosure. For example, the barrier may be injection-molded together during the manufacturing process of the lamp housing. In this way, according to the present disclosure, not only is there no need to add a separate component for the initial drive of the ultraviolet lamp, but also the structure of the barrier that induces the initial drive may be implemented very simply. As a result, the manufacturability of the far ultraviolet light emitting device may also be improved.

In addition, according to the present disclosure, since sparks are prevented even at high voltages, the maximum voltage that may be applied to the far ultraviolet light emitting device may be set high, and the voltage compatibility of the far ultraviolet light emitting device may also be improved.

In addition, in the present disclosure, the gap part formed between the barrier and the two electrode parts widens the surface area formed between the two electrode parts, thereby inducing a discharge between the two electrode parts. Through this, stable light emission of the ultraviolet lamp may be realized, and the quality of the far ultraviolet light emitting device may also be improved.

In the present disclosure, since the barrier provided in the lamp housing in the present disclosure acts as a dielectric, surface treatment for applying a dielectric to the surfaces of the two electrode parts is also unnecessary.

In addition, in the present disclosure, since the lighting of the ultraviolet lamp is induced through the gap part, a separate auxiliary light source for lighting is unnecessary, and thus, the far ultraviolet light emitting device has the effect of size reduction.

In particular, in the present disclosure, a first surface, a second surface, and a stepped surface between the first surface and the second surface may be formed on the surface of the electrode part. In this case, the distance (D1A) between the second surface and the surface of the barrier may be 0.8 to 1.2 times the distance (D2A) between the stepped surface and an electrode connection surface. In this way, the irradiation rate of the ultraviolet lamp may be increased while effectively preventing sparking.

In addition, the ultraviolet lamp of the present disclosure may have a surface light source structure. That is, since the surface of the ultraviolet lamp has a planar structure, the far ultraviolet light emitting device may provide a wider light extraction area. Through this, the luminous efficiency of the far ultraviolet light emitting device may also increase.

In particular, the ultraviolet lamp of the present disclosure has two electrode parts and the barrier in contact with each other on the electrode connection surface, and in the opposite surface, the light extraction surface of the ultraviolet lamp may be exposed without being covered by other parts. Since the exposure area of the light extraction surface is widened in this way, the luminous efficiency of the far ultraviolet light emitting device may be further improved.

In addition, the present disclosure may be applied not only to a flat-type lamp made by sealing two flat plates, but also to a tube-type lamp. In this way, the present disclosure has the advantage of high compatibility because it may be applied to all types of ultraviolet lamps.

In addition, in the present disclosure, the electrode part may be equipped with an auxiliary electrode protruding toward the barrier. This auxiliary electrode may concentrate the electric field of the discharge through the protruding shape, and accordingly, the ultraviolet lamp may be started more effectively.

### BRIEF DESCRIPTION OF THE DRAWINGS

FIG. 1 is a perspective view showing an air conditioner incorporating an embodiment of a far ultraviolet light emitting device according to the present disclosure.

FIG. 2 is an exemplary view illustrating a state in which light rays are irradiated in an interior space in which the far ultraviolet light emitting device according to the present disclosure is installed.

FIG. 3 is a perspective view showing the appearance of the air conditioner to which the embodiment of the present disclosure is applied.

FIG. 4 is an enlarged perspective view of a part of FIG. 3.

FIG. 5 is a perspective view showing the structure of a far ultraviolet light emitting module to which the embodiment of the present disclosure is applied.

FIG. 6 is a perspective view showing the structure of the far ultraviolet light emitting module to which the embodiment of the present disclosure is applied from an angle different from that of FIG. 5.

FIG. 7 is an exploded perspective view showing components of the far ultraviolet light emitting module to which the embodiment of the present disclosure is applied.

FIG. 8 is a perspective view of the far ultraviolet light emitting device according to the embodiment of the present disclosure.

FIG. 9 is an exploded perspective view of components constituting the embodiment of the far ultraviolet light emitting device according to the present disclosure.

FIG. 10 is a perspective view showing a state in which the ultraviolet lamp is omitted from FIG. 8.

FIG. 11 is a plan view showing a state in which the ultraviolet lamp is omitted from FIG. 8.

FIG. 12 is a perspective view showing a state in which a pair of electrode parts are disassembled in a lamp housing constituting the embodiment of the present disclosure.

FIG. 13 is an enlarged perspective view of a barrier of the lamp housing constituting the embodiment of the present disclosure.

FIG. 14 is a cross-sectional view of the line XIV-XIV′ of FIG. 12.

FIG. 15 is a plan view showing a structure of a pair of electrode parts constituting the embodiment of the present disclosure.

FIG. 16 is a perspective view showing a structure of a first electrode part constituting the embodiment of the present disclosure.

FIG. 17 is a side view showing the structure of a pair of electrode parts constituting the embodiment of the present disclosure.

FIG. 18 is a cross-sectional view of the line XVIII-XVIII′ of FIG. 10.

FIGS. 19 to 25 are cross-sectional views showing various modified embodiments of a pair of electrode parts and barriers constituting the present disclosure.

FIG. 26 and FIG. 27 are plan views showing other embodiments of an auxiliary electrode provided in a pair of electrode parts constituting the present disclosure.

FIG. 28 is a perspective view of the far ultraviolet light emitting device according to another embodiment of the present disclosure.

FIG. 29 is a cross-sectional view showing the structure of the embodiment shown in FIG. 28.

FIG. 30 is a perspective view showing a pair of electrode parts constituting the embodiment shown in FIG. 28.

FIG. 31 is a perspective view showing another embodiment of an electrode part constituting the far ultraviolet light emitting device according to the present disclosure.

FIG. 32 is a perspective view of a far ultraviolet light emitting device according to another embodiment of the present disclosure.

FIG. 33 is a plan view showing the structure of the embodiment shown in FIG. 32.

FIG. 34 is a perspective view showing a structure of a pair of electrode parts and the lamp housing constituting the embodiment shown in FIG. 32.

FIG. 35 is a plan view showing the structure of a pair of electrode parts and the lamp housing constituting the embodiment shown in FIG. 32.

FIG. 36 is a cross-sectional view of the line XXXVI-XXXVI′ of FIG. 35.

FIG. 37 is a cross-sectional view of the line XXXVII-XXXVII′ of FIG. 35

FIG. 38 is a perspective view showing the structure of the lamp housing constituting the embodiment shown in FIG. 33.

FIG. 39 is a plan view showing the structure of the lamp housing constituting the embodiment shown in FIG. 33.

FIG. 40 is a cross-sectional view of the line XL-XL′ of FIG. 38.

FIG. 41 is a perspective view showing the structure of a pair of electrode parts constituting the embodiment shown in FIG. 33.

FIG. 42 is a cross-sectional view showing a structure of the pair of electrode parts constituting the embodiment shown in FIG. 33.

FIG. 43 is a perspective view showing the structure of the first electrode part constituting the embodiment shown in FIG. 33.

FIG. 44 is a cross-sectional view of the line XLIV-XLIV′ of FIG. 33.

FIG. 45 is a cross-sectional view of the line XXXVI-XXXVI′ of FIG. 35.

FIG. 46 is a cross-sectional view of the line XLVI-XLVI′ of FIG. 33.

FIGS. 47-50 illustrates another embodiment of the far ultraviolet light emitting device or light source.

### MODE FOR INVENTION

Hereinafter, some embodiments of the present disclosure will be described in detail through exemplary drawings. In adding reference numerals to the components of each drawing, it should be noted that the same components have the same numerals as possible even if they are displayed on different drawings. In addition, in describing an embodiment of the present disclosure, if it is determined that a detailed description of a related known configuration or function hinders understanding of an embodiment of the present disclosure, the detailed description thereof will be omitted.

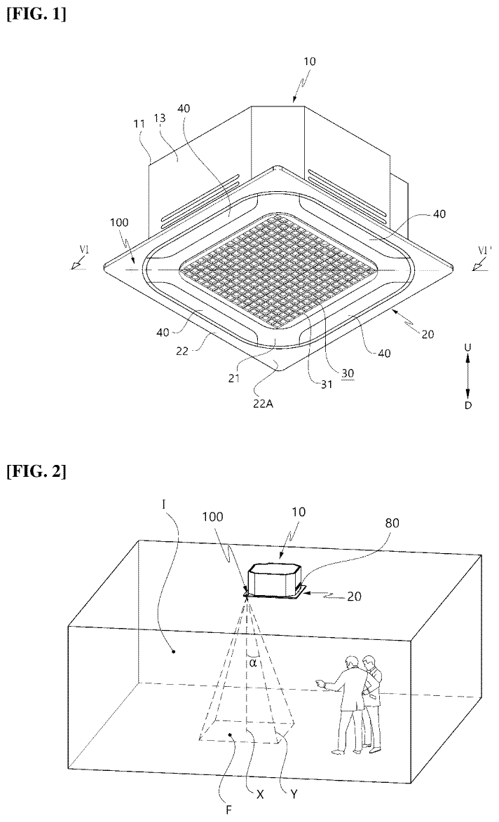

A far ultraviolet light emitting device (C) of present disclosure may be used for sterilization. For example, the far ultraviolet light emitting device (C) may be coupled to a home appliance to provide a user with a sterilizing function as well as an original function of the home appliance. For example, the ultraviolet sterilization device of the present disclosure may be applied to an air conditioner. In FIG. 1, an exemplary view is shown that the ultraviolet sterilization device according to the present disclosure is applied to an air conditioner. As another example, the ultraviolet sterilization device of the present disclosure may be applied not to an air conditioner, but to various home appliances such as an air purifier, a dish dryer, a dishwasher, an air conditioner, a refrigerator, a kimchi refrigerator, a water purifier, a microwave oven, a washing machine, a dryer, a clothing steamer, a cleaning robot, a lighting, a shoe treatment device, and a pet appliance.

The far ultraviolet light emitting device (C) according to the present disclosure may be modularized and applied to home appliances. For example, the far ultraviolet light emitting module 100 may include a component for supplying power to the far ultraviolet light emitting device (C), and the ray generating apparatus far ultraviolet light emitting device (C) may include a component for mounting to home appliances or the like. Hereinafter, the far ultraviolet light emitting module 100 will be described as a concept including a far ultraviolet light emitting device, an inverter device 150, and a device frame 110. The following mainly described a structure of the far ultraviolet light emitting device (C) provided in an air conditioner as an example among the home appliances.

Referring to FIG. 2, the far ultraviolet light emitting module 100 may irradiate ultraviolet light toward an interior (I) place where the air conditioner is installed. Here, the interior (I) means the place where the air conditioner is installed, and means the space where the air conditioner aims to control temperature or humidity.

Referring to FIG. 1, the appearance of the air conditioner is illustrated. The air conditioner may be installed on a ceiling. When the air conditioner is installed on the ceiling, a part of the air conditioner may be contained inside the ceiling, and the remaining part may be exposed to the interior (I). More precisely, among casings 10, 20 described below, an upper casing 10 may be contained inside the ceiling, and a lower casing 20 may be exposed to the interior (I). For reference, in the drawing, U means direction toward the ceiling, and D means direction toward the interior (I).

The overall shape of the upper casing 10 among the casings 10, 20 may be approximately a rectangular solid shape. The upper casing 10 may include a base plate 11 arranged inside the ceiling, and a side plate 13 erected along the edge of the base plate 11.

The lower casing 20 may be combined with the upper casing 10. The lower casing 20 may cover the open bottom surface of the upper casing 10. Here, the bottom surface of the lower casing 20 is based on the direction shown in FIG. 1. That is, the lower casing 20 may be arranged to face the floor of the interior (I). The lower casing 20 may be combined with the upper casing 10 as a separate object from the upper casing 10. As another example, the lower casing 20 may be provided as an integral part with the upper casing 10.

The lower casing 20 may include an inner plate 21 and an outer plate 22. The inner plate 21 may form the center of the lower casing 20. An air inlet 30 may be provided at the center of the inner plate 21. An air outlet may be provided at an outer part of the inner plate 21. The outer plate 22 may be arranged around the periphery of the inner plate 21. The reference numeral 31 is a grille part forming the air inlet 30.

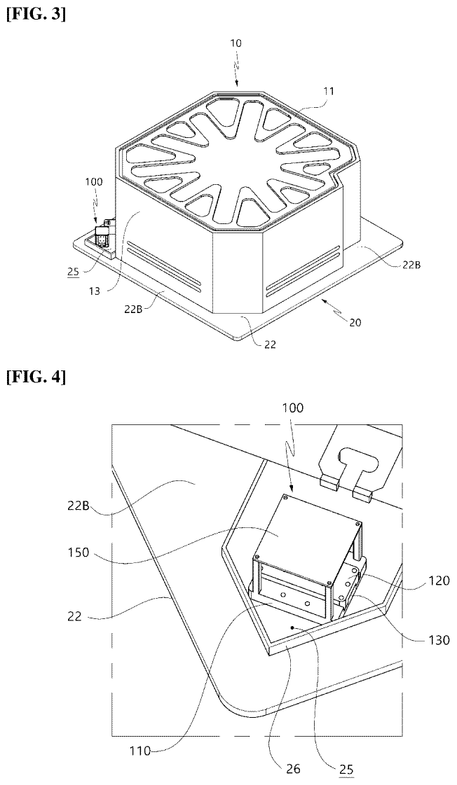

A part of the surface of the lower casing 20 may be exposed to the interior (I). Referring to FIGS. 1 and 2, a first plate surface 22A, which is the bottom surface of the lower casing 20, is exposed to the interior (I). In the air conditioner, a portion positioned above the first plate surface 22A of the lower casing 20 may be contained in the ceiling and not exposed. For example, as shown in FIG. 3, a second plate surface 22B opposite the first plate surface 22A of the lower casing 20 faces the inside of the ceiling and is therefore not exposed to the interior (I). Referring to FIG. 2, the upper casing 10 is shown contained inside the ceiling.

As shown in FIG. 2, the far ultraviolet light emitting module 100 arranged in the lower casing 20 may ultraviolet light toward the interior (I). In FIG. 2, α represents ½ of the light irradiation angle irradiated by the far ultraviolet light emitting module 100. The ultraviolet light irradiated in this manner reaches the ground of the interior (I), and the reference symbol X represents the shortest distance between the light irradiated from the far ultraviolet light emitting module 100 and the ground of the interior (I). Since the ultraviolet light irradiated from the far ultraviolet light emitting module 100 is spread at an angle α, it may have a sterilization range (F) surrounded by the reference symbol Y The inside of the sterilization range (F) may be sterilized by the far ultraviolet light emitting module 100.

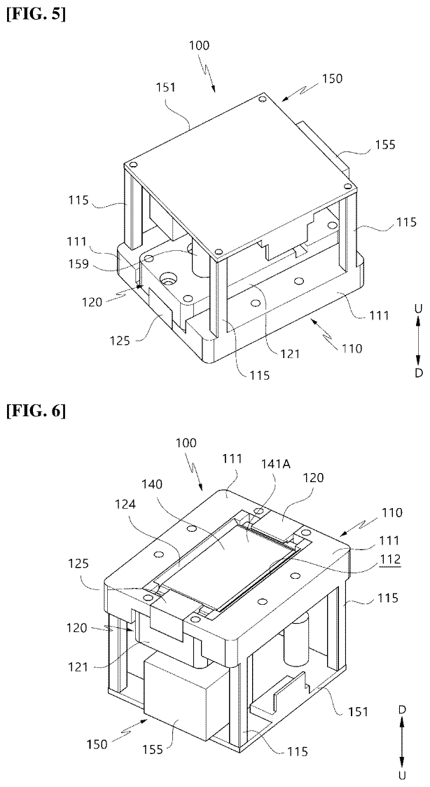

Referring to FIGS. 4 and 5, the far ultraviolet light emitting module 100 is disposed in the lower casing 20. More precisely, the far ultraviolet light emitting module 100 may be placed in a mounting part 25 provided in the lower casing 20. Since the mounting part 25 is a portion that is not exposed to the interior (I), most of the far ultraviolet light emitting module 100 is covered in the interior (I). However, a portion of the far ultraviolet light emitting module 100 is placed so as to face the interior (I), and the portion of the far ultraviolet light emitting module 100 facing the interior (I) may emit ultraviolet light, thereby sterilizing the interior (I). The reference numeral 26 indicates a mounting fence surrounding the mounting part 25.

Next, the far ultraviolet light emitting module 100 will be described in detail. The far ultraviolet light emitting module 100 may irradiate far ultraviolet light to the outside of the casing 10, 20. More precisely, the far ultraviolet light emitting module 100 is arranged in the casing 10, 20 so as to face the interior (I), and irradiates far ultraviolet light into the interior (I). Through this, the far ultraviolet light emitting module 100 may sterilize the interior (I).

The ultraviolet light irradiated by the ultraviolet lamp 140 constituting the far ultraviolet light emitting module 100 may be subdivided according to the length of the wavelength. For example, the ultraviolet light may be divided into UV-A with a wavelength of 320 nm to 400 nm, UV-B with a wavelength of 280 nm to 320 nm, and UV-C with a wavelength of 200 nm to 280 nm. Among these, UV-C, which is a short-wavelength ultraviolet, has the characteristic of breaking down bacteria DNA and causing a chemical reaction with certain substances, making it an effective tool for sterilization.

In this embodiment, the ultraviolet lamp 140 irradiates UV-C, more precisely, it may generate far ultraviolet rays (far-UVC) that are a narrow spectrum within UV-C light. Far-UVC are known to provide the same pathogen-killing effect as UV-C light, but without the harmful side effects of other frequencies or wavelengths. The far ultraviolet light emitting module 100 may utilize excimer discharge to generate far-UVC of a specific wavelength range.

The ultraviolet lamp 140 of the present disclosure may use a far UV-C excimer lamp that may affect a long distance of about 2.5m, and may sterilize not only the air but also the furniture, floors, and walls within the space. However, the UV-C excimer type ultraviolet lamp 140 also includes a small amount of light in other frequency bands, and a wavelength of 238 nm or more is harmful to a human body, and thus an optical filter (not shown) for removing ultraviolet ray having the wavelength of 238 nm or more may be further provided. The optical filter may transmit 80% or more of ultraviolet ray having 200 to 238 nm harmless to a human body, and transmit less than 5% of ultraviolet ray having 238 nm to 280 nm harmful to a human body. As another example, the optical filter may be omitted.

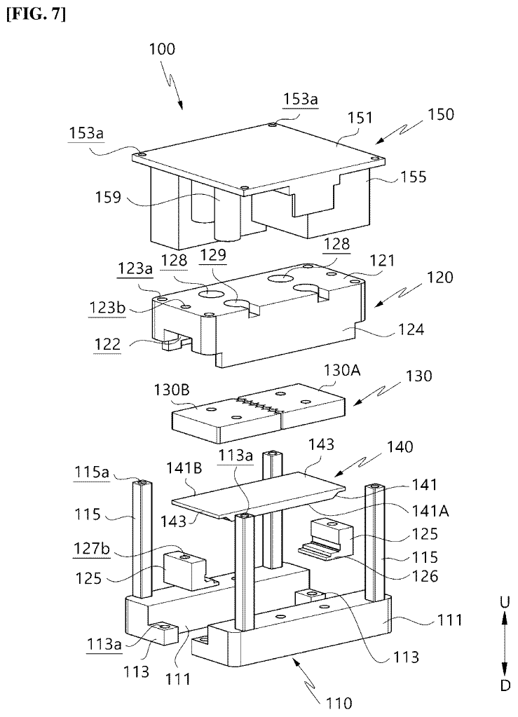

Referring to FIGS. 5 to 7, the far ultraviolet light emitting module 100 includes a device frame 110 fixed to the casings 10, 20. The device frame 110 may allow the far ultraviolet light emitting module 100 to be secured to the casings 10, 20 while forming the skeleton of the far ultraviolet light emitting module 100. As shown in FIG. 7, the device frame 110 may include a pair of frames spaced apart from each other. A lamp housing 120 is disposed between the pair of frames.

The device frame 110 may include a support part 111 in close contact with the casings 10 and 20, and a post part 115 erected at the support part 111. Referring to FIG. 7, a bottom surface of the support part 111 may be in close contact with a second plate surface 22B of the outer casings 10 and 20, and a pair of post parts 115 may extend from a top surface of the support part 111. An inverter device 150 may be mounted on an upper end of the post part 115. A post hole 115a may be formed in the post part 115. The post hole 115a may be connected to an inverter hole 153a of the inverter device 150, and when a fastener (not shown) such as a bolt is fastened to the inverter hole 153a and the post hole 115a, the inverter device 150 may be fixed to the post part 115.

A fixing end 113 may be provided on the support part 111. The fixing end 113 may extend in a direction of increasing the area of the bottom surface of the support part 111. In the embodiment, the fixing end 113 protrudes in a direction of narrowing the gap between the pair of device frames 110. The lamp housing 120 may be disposed above the fixing end 113. A first fixing hole 113a formed in the fixing end 113 may be connected to a second fixing hole 123a provided in the lamp housing 120 and fastened thereto through a separate fastener (not shown).

The lamp housing 120 is disposed in the device frame 110. The ultraviolet lamp 140 and a pair of electrode parts 130 (130A and 130B) are accommodated in the lamp housing 120. A housing body 121 forming the frame of the lamp housing 120 has a substantially hexahedral shape, but may be deformed according to the shape of the electrode part 130 and the ultraviolet lamp 140. A mounting space 122 may be recessed in the center of the housing body 121. The electrode part 130 and the ultraviolet lamp 140 may be stacked in the mounting space 122.

The second fixing hole 123a corresponding to the first fixing hole 113a formed in the fixing end 113 may be formed in the housing body 121 to be fastened thereto. As another example, the second fixing hole 123a may be omitted, and the housing body 121 may be press-fitted into the device frame 110 or may be hooked and secured to the device frame 110. Reference numeral 123b is a holder assembly hole formed in the housing body 121, and an irradiation holder 125 to be described later may be fastened to the holder assembly hole 123b.

Referring to FIG. 7, the housing body 121 may include a storage fence 124. The storage fence 124 may protrude from an edge of the mounting space 122 to surround both sides of the mounting space 122. The mounting space 122 may be disposed between a pair of the storage fences 124. Referring to FIG. 9, an inclined portion 124a may be provided at an upper portion of the storage fence 124 to help mounting of the electrode part 130 and the ultraviolet lamp 140, and may reflect ultraviolet light emitted from the ultraviolet lamp 140.

The lamp housing 120 may include the irradiation holder 125. The irradiation holder 125 may be assembled with the housing body 121 to hang a portion of the ultraviolet lamp 140. The irradiation holder 125 may secure the ultraviolet lamp 140 and the electrode part 130 disposed above the ultraviolet lamp 140 (based on FIG. 7). The irradiation holder 125 is separate from the housing body 121 and may be fixed with a separate fastener (not shown). Reference numeral 127b is a counter fastening hole formed in the irradiation holder 125, and the relative fastening hole 127b is connected to the holder assembly hole and fastened thereto.

The irradiation holder 125 may include a holder body including the counter fastening hole 127b and a hook arm 126 extending from the holder body to secure the ultraviolet lamp 140. The hook arm 126 may extend in a direction orthogonal to the holder body and may be disposed on a light extraction surface 141A of the ultraviolet lamp 140.

As shown in FIG. 7, a power connection hole 128 may be formed in the housing body 121. The inverter device 150 may be connected to the power connection hole 128. A part of the inverter device 150 may be directly inserted into the power connection hole 128 to supply AC power. As another example, a wire or a terminal extending from the inverter device 150 may be inserted into the power connection hole 128. As another example, the inverter device 150 may be omitted, and a wire or a terminal for applying power from the outside may be inserted into the power connection hole 128. Reference numeral 129 is an inverter fixing groove for fixing the inverter device 150, and a fixing protrusion 159 of the inverter device 150 is inserted into the inverter fixing groove 129.

The electrode part 130 may be disposed on the lamp housing 120. The electrode part 130 is provided to supply power to the ultraviolet lamp 140. The electrode part 130 may receive AC power from the inverter device 150 and transmit the received AC power to the ultraviolet lamp 140, thereby inducing discharge of the ultraviolet lamp 140. The electrode part 130 may include a pair of electrode parts 130A and 130B.

AC power may be applied to the pair of electrode parts 130A and 130B from the inverter device 150, respectively. Since the pair of electrode parts 130A and 130B which have received AC power are in contact with the ultraviolet lamp 140, respectively, they may induce discharge of an inert gas filled inside the ultraviolet lamp 140. The pair of electrode parts 130A and 130B are each made of a conductive material. In the embodiment, the pair of electrode parts 130A and 130B have a block shape.

In the embodiment, the ultraviolet lamp 140 generates a far-UVC. The ultraviolet lamp 140 may have an approximately plate-shaped structure. The ultraviolet lamp 140 supports the electrode part 130 and transmits far-UVC, and may be generally made of quartz or ceramic materials that have good ultraviolet ray transmittance. Alternatively, the ultraviolet lamp 140 may be made of fused silica having an OH content less than that of quartz and thus excellent ultraviolet ray transmittance.

The ultraviolet lamp 140 may be formed of a plurality of plate members, and a discharge space may be formed between the plate members. The plurality of plate members may be bonded through melting, and the discharge space may be formed therebetween. In the embodiment, the ultraviolet lamp 140 may be configured as an excimer lamp. The reference numeral 143 is a side end portion provided at both ends of the ultraviolet lamp 140 in the longitudinal direction, and the side end portion 143 may be viewed as a portion melted and sealed.

For reference, the longitudinal direction of the ultraviolet lamp 140 is the same as the direction in which the pair of electrode parts 130A, 130B are spaced from each other. The longitudinal direction of the ultraviolet lamp 140 is orthogonal to the longitudinal direction of a barrier 126 (up and down direction of FIG. 11).

The discharge space formed inside the ultraviolet lamp 140 may be equipped with an inert gas selected from the group consisting of argon (Ar), neon (Ne), xenon (Xe), and krypton (Kr). In this case, the inert gas may be selected from the group consisting of ArBr, ArCl, ArF, ArO, NeF, XeI, XeO, XeBr, XeCl, XeF, KrBr, KrCl, KrO, and KrF.

Far UVC rays are generated and radiated by the discharge of the ultraviolet lamp 140. In this case, the wavelength of the radiated ray may vary depending on the type of inert gas. In this embodiment, the ultraviolet lamp 140 generates a wavelength of 235 to 260 nm, and may remove foodborne pathogens, natural microorganisms, molds, yeast, etc.

At least a part of the light extraction surface 141A of the ultraviolet lamp 140 may be arranged to face the interior (I). Referring to FIG. 2, the ultraviolet lamp 140 may irradiate far UVC downward. In this embodiment, since the air conditioner is installed on the ceiling, the light extraction surface 141A of the ultraviolet lamp 140 may be arranged to face the ground of the interior (I). Reference numeral 141B indicates an electrode connection surface 141B formed on the opposite side of the light extraction surface 141A of the ultraviolet lamp 140.

The inverter device 150 may supply AC power to the pair of electrode parts 130. The inverter device 150 may be arranged on the opposite side of the ultraviolet lamp 140 with the pair of electrode parts 130 as the center. The inverter device 150 may include an inverter body 151 and an AC generator 155 provided in the inverter body 151. The inverter device 150 may include a converter part that converts commercial power from the air conditioner into DC power, and an inverter part that removes ripples in a smoothing circuit and then converts it back into AC. As another example, the inverter device 150 may be omitted or arranged apart from the far ultraviolet light emitting module 100.

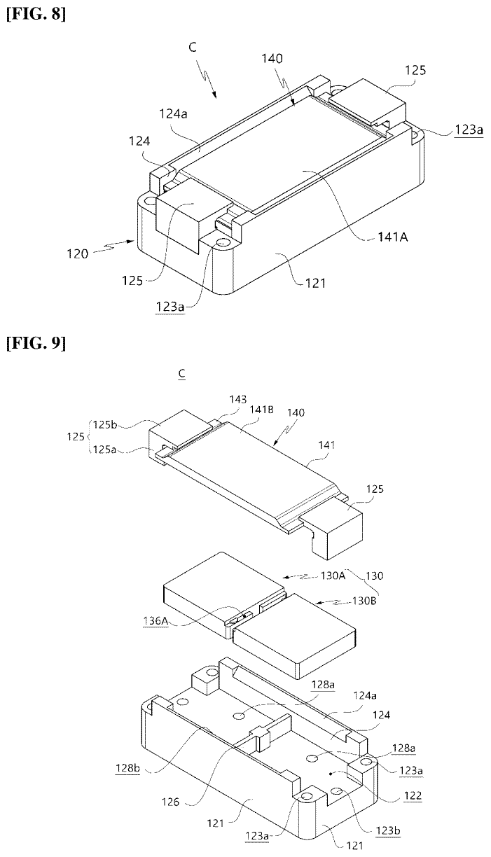

Referring to FIGS. 8 and 9, a far ultraviolet light emitting device (C) is shown according to the embodiment of the present disclosure. Here, it may be considered that the far ultraviolet light emitting device (C) includes the lamp housing 120, the pair of electrode parts 130A and 130B, and the ultraviolet lamp 140 except for the mounting frame 110 and the inverter device 150. For reference, in FIGS. 8 and 9, the light extraction surface 141A of the ultraviolet lamp 140 is shown facing upward.

As shown in FIG. 9, the mounting space 122 is provided in the housing body 121 of the lamp housing 120. The power connection hole 128 described above is formed on the bottom surface of the mounting space 122, and the power connection hole 128 may include a pair of power connection holes 128a and 128b. In this case, the pair of power connection holes 128a and 128b may be formed on both sides with respect to the barrier 126, respectively. A pair of power connection holes 128a and 128b formed on both sides may supply power to the first electrode part 130A and the second electrode part 130B constituting the pair of electrode parts 130A and 130B, respectively. To this end, the pair of power connection holes 128a and 128b may be connected to the inverter device 150 or an external power supply device.

The inclined portion 124a may be formed on the storage fence 124 of the housing body 121. The inclined portion 124a may have a structure inclined downward toward the mounting space 122. The inclined portion 124a may reflect the far UVC emitted from the ultraviolet lamp 140 to increase the efficiency of the far ultraviolet light emitting device (C). In the present embodiment, the inclined portion 124a may be formed to have a height facing the side surface of the ultraviolet lamp 140.

The barrier 126 is provided in the mounting space 122. The barrier 126 may be erected on a bottom surface of the mounting space 122. Here, the bottom surface refers to a surface on which each of the pair of electrode parts 130A and 130B is seated. The barrier 126 may be disposed between the pair of electrode parts 130A and 130B to separate the pair of electrode parts 130A and 130B from each other. To this end, the barrier 126 may be disposed across the mounting space 122. More specifically, the barrier 126 may be continuously formed along a direction orthogonal to a direction in which the first electrode part 130A and the second electrode part 130B face each other.

The barrier 126 may be arranged between the pair of electrode parts 130A, 130B and may become a type of dielectric. When the barrier 126 functions as a dielectric, the barrier 126 together with the pair of electrode parts 130A, 130B may become a type of capacitor. This capacitor structure may assist in starting the ultraviolet lamp 140. More specifically, the electric polarization phenomenon by the barrier 126 may be utilized to induce discharge (lighting up) of the ultraviolet lamp 140 arranged above the barrier 126.

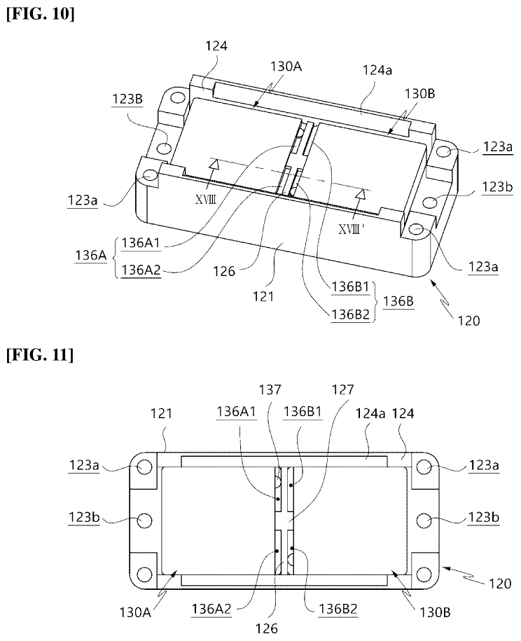

In this case, as will be described below, the far ultraviolet light emitting device (C) of the present embodiment is provided with a gap part 136, (see FIG. 10), and the discharge electric field may be concentrated in the gap part 136. That is, the electric field is strengthened as the dielectric molecules of the barrier 126, which is a dielectric, are polarized. This concentration of the discharge electric field may be transmitted to the surface of the ultraviolet lamp 140 to realize effective starting.

The dielectric constant of the barrier 126 may have a range of 1 to 15. In the present embodiment, the barrier 126 is integrally provided with the lamp housing 120, so the dielectric constant of the barrier 126 is the same as the dielectric constant of the lamp housing 120. The lamp housing 120 may have various materials such as plastic, silicone, rubber, etc. In another example, the barrier 126 may be configured as a separate object from the lamp housing 120 and may be assembled to the lamp housing 120.

Referring at FIG. 10 and FIG. 11, the first electrode part 130A and the second electrode part 130B may be arranged on both sides of the barrier 126 as the center. When the first electrode part 130A and the second electrode part 130B are stored in the mounting space 122, the barrier 126 naturally blocks the space between the first electrode part 130A and the second electrode part 130B. In addition, the upper portions of the first electrode part 130A, the barrier 126, and the second electrode part 130B are open, and the ultraviolet lamp 140 may be seated thereon.

As illustrated, the gap part 136 may be provided on the upper portions of the first electrode part 130A and the barrier 126 and the upper portions of the second electrode part 130B and the barrier 126, respectively. The gap part 136 may be formed such that at least one of the surfaces of the first electrode part 130A or the second electrode part 130B is spaced apart from the surface of the barrier 126. The gap part 136 is opened upward, and in the present embodiment, the gap part 136 may be opened toward the electrode connection surface 141B. This gap part 136 may prevent sparking between the first electrode part 130A and the second electrode part 130B and may also assist in starting the ultraviolet lamp 140 through electric field concentration. The gap part 136 will be described again below.

Referring to FIG. 11, the barrier 126 may be provided with a barrier protrusion 127. The barrier protrusion 127 protrudes from the surface of the barrier 126, but protrudes in the direction of the surface of the first electrode part 130A and the surface of the second electrode part 130B. The barrier protrusion 127 may be said to be a portion where the thickness of the barrier 126 becomes relatively large. The barrier protrusion 127 may be provided at the center of the barrier 126, or may be provided at a position biased from the center of the barrier 126 to the storage fence 124. As another example, the barrier protrusion 127 may be provided so as to protrude only in one direction among the surface of the first electrode part 130A or the surface of the second electrode part 130B.

As described below, the first electrode part 130A and the second electrode part 130B may each have an electrode groove 135′, (see FIG. 16) formed at a position corresponding to the barrier protrusion 127. The electrode groove 135′ of the first electrode part 130A will be referred to as a first electrode groove 135′, and the electrode groove 135′ of the second electrode part 130B will be referred to as a second electrode groove (not given a reference numeral).

The barrier protrusions 127 may each be arranged in the first electrode groove 135′ and the second electrode groove. The two first electrode protrusions 135A of the first electrode part 130A to be described below may be divided in the left and right directions based on the first electrode groove 135′. In addition, the two second electrode protrusions 135B of the second electrode part 130B to be described below may be divided in the left and right directions based on the first electrode groove 135′.

The barrier protrusion 127 may extend from the bottom surface of the lamp housing 120 toward the electrode connection surface 141B. The first electrode groove 135′ and the second electrode groove may extend from the bottom surface 133A of the first electrode part 130A and the bottom surface 133B of the second electrode part 130B that contact the bottom surface of the lamp housing 120, respectively, toward the electrode connection surface 141B. Accordingly, the barrier protrusion 127 and the first electrode groove 135′ and the second electrode groove may be formed in a direction parallel to each other.

When the barrier protrusions 127 are disposed in the first electrode groove 135′ and the second electrode groove, respectively, the first electrode part 130A and the second electrode part 130B may be aligned with the lamp housing 120. Also, the barrier protrusion part 127 may increase the surface area of the barrier 126 to further activate the electro-polarization phenomenon.

The pair of electrode parts 130A and 130B may have different polarities. The pair of electrode parts 130A and 130B may receive AC power from the inverter device 150 to emit light of the ultraviolet lamp 140. Each of the first electrode part 130A and the second electrode part 130B constituting the pair of electrode parts 130A and 130B may have an approximately hexahedral shape. In the present embodiment, each of the first electrode part 130A and the second electrode part 130B has a shape having a relatively larger front, rear, and left and right widths than a height thereof.

At least one of the surfaces of the first electrode part 130A and the second electrode part 130B may be formed to be spaced apart from the surface of the barrier 126 to form the gap part 136. The gap part 136 may be opened toward the electrode connection surface 141B. The gap part 136 may separate between the surface of the first electrode part 130A and the surface of the barrier 126 and/or between the surface of the second electrode part 130B and the surface of the barrier 126.

The gap part may be covered by the ultraviolet lamp 140 to be a predetermined empty space. The gap part 136 may prevent a spark caused by a high voltage when power is applied to the first electrode part 130A and the second electrode part 130B. Also, since the gap part 136 is formed of a dielectric material, it is possible to induce the start of the ultraviolet lamp 140 while discharging. That is, the discharge electric field is concentrated in the gap part 136 which is a kind of stepped space due to the dielectric constant of the barrier 126, and is transmitted to the surface of the ultraviolet lamp 140 to initiate the starting process.

The gap part 136 may be continuous in a direction in which the barrier 126 is provided across the lamp housing 120 (vertical direction based on FIG. 11). The continuous gap part 136 allows a predetermined empty space to be continuous between the pair of electrode parts 130A and 130B and the barrier 126, so that spark prevention and start of the ultraviolet lamp 140 may be stably performed.

In the present embodiment, since the barrier protrusion 127 is provided at the center of the barrier 126, the gap part 136 may be divided into two parts with respect to the barrier protrusion 127. More specifically, a first gap part 136A formed between the surface of the first electrode part 130A and a first barrier surface 126A1 may be divided into two parts. A second gap part 136B formed between the surface of the second electrode part 130B and a second barrier surface 126A2 may be divided into two parts. Hereinafter, two gap parts constituting the first gap part 136A are divided into a 1-1 gap part 136A1 and a 1-2 gap part 136A2, respectively, and two gap parts constituting the second gap part 136B are divided into a 2-1 gap part 136B1 and a 2-2 gap part 136B2, respectively.

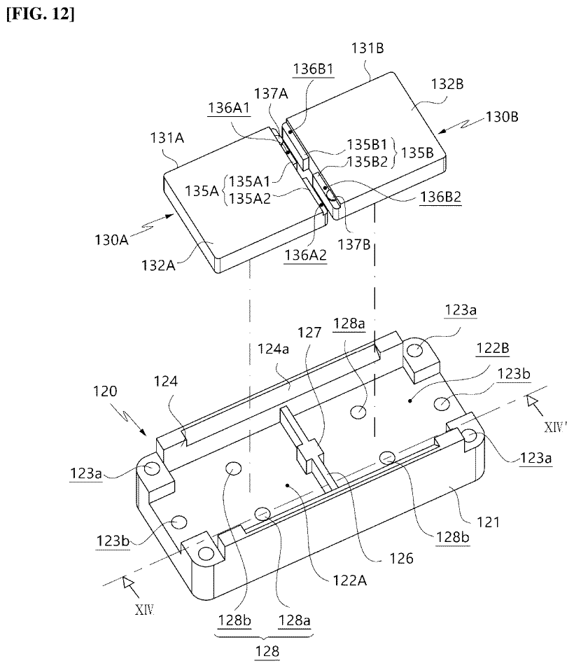

Referring to FIG. 12, the pair of electrode parts 130A and 130B are disassembled from the lamp housing 120. The pair of electrode parts 130A and 130B are disposed to face each other. The barrier 126 is disposed between the pair of electrode parts 130A and 130B facing each other. When the barrier 126 is disposed between the pair of electrode parts 130A and 130B, surfaces of the pair of electrode parts 130A and 130B are covered by the barrier 126. Then, the gap parts 136A and 136B are formed between the pair of electrode parts 130A and 130B and the barrier 126.

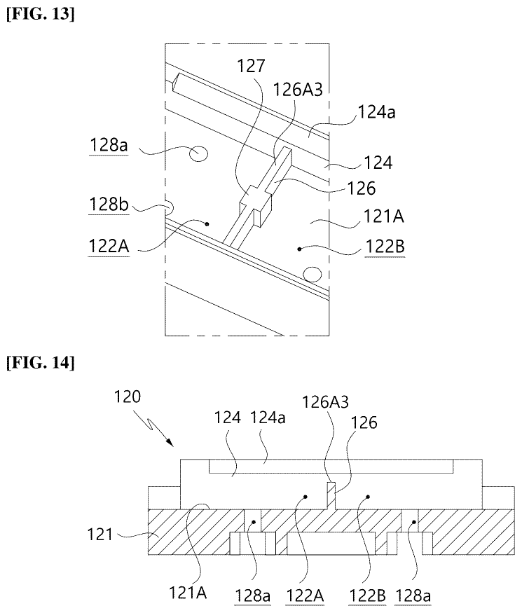

Referring to FIG. 13, the barrier 126 is disposed to cross the mounting space 122. Accordingly, the mounting space 122 may be divided into a first mounting space 122A and a second mounting space 122B. The first electrode part 130A is disposed in the first mounting space 122A, and the second electrode part 130B is disposed in the second mounting space 122B. Since both end portions of the barrier 126 are connected to both end portions of the mounting space 122, the barrier 126 may completely cross the mounting space 122. The barrier protrusion 127 may protrude toward the first mounting space 122A and the second mounting space 122B, respectively.

Referring to FIG. 14, a side cross-sectional view of the lamp housing 120 is illustrated. The barrier 126 may be erected in the lamp housing 120. Here, the erection of the barrier 126 means that the barrier 126 is provided in a vertical direction with respect to FIG. 14, and a base portion of the barrier 126 is formed on a bottom surface of the mounting space. The upper end of the barrier 126 may be formed to be lower than the upper end of the storage fence 124. The ultraviolet lamp 140 may be disposed between the upper end of the barrier 126 and the upper end of the storage fence 124.

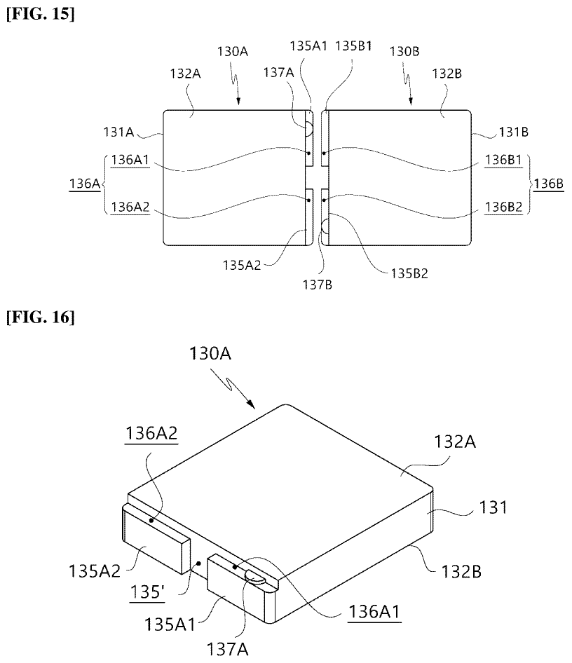

Referring to FIGS. 12 and 15, the pair of electrode parts 130A and 130B are disposed to face each other. As shown above, the pair of electrode parts 130A and 130B may have structures symmetrical to each other. Electrode protrusions 135A and 135B may be provided on surfaces of the pair of electrode parts 130A and 130B facing each other, respectively. The electrode protrusions 135A and 135B may be formed to protrude from surfaces of the pair of electrode parts 130A and 130B facing each other toward the surface of the barrier. The structure will be described again below.

The pair of electrode parts 130A, 130B have a structure that is symmetrical to each other. The pair of electrode parts 130A, 130B will be described with reference to FIG. 16, with the first electrode part 130A as a reference. The first electrode part 130A has an approximately hexahedral shape. With reference to FIG. 16, an upper surface 132A of the first electrode part 130A may be in contact with the electrode connection surface 141B of the ultraviolet lamp 140. Since the upper surface 132A of the first electrode part 130A has a planar structure, it may also be in surface contact with an electrode connection surface 141B of the ultraviolet lamp 140. A lower surface 133A of the first electrode part 130A is in close contact with a first mounting space 122A. The lower surface 133A of the first electrode part 130A is connected to the inverter device 150 through the power connection hole 128a, 128b and may receive power.

A first electrode protrusion 135A may be formed on a surface of the first electrode part 130A between the upper surface 132A of the first electrode part 130A and the lower surface 133A of the first electrode part 130A. The first electrode protrusion 135A protrudes from the front surface of the first electrode part 130A, which is facing the surface of the barrier 126. The first electrode protrusion 135A does not occupy the entire front surface of the first electrode part 130A, but rather occupies a part of the front surface of the first electrode part 130A. Accordingly, the portion where the first electrode protrusion 135A protrudes may form a stepped structure with the remaining portion. Among these stepped structures, the stepped structure facing the ultraviolet lamp 140 has the first gap part 136A formed therein. The first gap part 136A is formed by the first electrode part 130A and the barrier 126 together, but for ease of understanding, the gap part 136 is expressed in the drawing as being formed in the first electrode part 130A.

The first electrode protrusion 135A may be formed as a pair. In the present embodiment, the first electrode protrusion 135A is formed by a 1-1 electrode protrusion 135A1 and a 1-2 electrode protrusion 135A2 that are spaced apart from each other. Afirst electrode groove 135′ in which the barrier protrusion 127 is arranged may be formed between the 1-1 electrode protrusion 135A1 and the 1-2 electrode protrusion 135A2. The first electrode groove 135′ may be seen as having a relatively recessed structure compared to the 1-1 electrode protrusion 135A1 and the 1-2 electrode protrusion 135A2.

The 1-1 gap part 136A1 is formed in a relatively recessed portion at the upper portion of the 1-1 electrode protrusion 135A1. In FIG. 16, the 1-1 gap part 136A1 is open and exposed, but the 1-1 gap part 136A1 may be shielded by the surface of the barrier 126 and the surface of the ultraviolet lamp 140.

In this case, the separation space may be formed on any one surface between the surface of the barrier 126, the electrode connection surface 141B, and the surface of the first electrode part 130A, or between the surface of the barrier 126, the electrode connection surface 141B, and the surface of the second electrode part 130B. The structure of this gap part 136, (separation space) will be described again in detail below.

The 1-2 gap part 136A2 is formed in a relatively recessed portion on the upper portion of the 1-2 electrode protrusion 135A2. The 1-2 gap part 136A2 is adjacent to the 1-1 gap part 136A1, and the first electrode groove 135′ is arranged between the 1-1 gap part 136A1 and the 1-2 gap part 136A2. The 1-2 gap part 136A2 may be shielded by the surface of the barrier 126 and the surface of the ultraviolet lamp 140. The shielded space may be viewed as a separation space.

The separation space may be viewed as being formed by the first electrode part 130A or the second electrode part 130B being separated from the electrode connection surface 141B in the vertical direction. In this case, the separation space may be connected to the surface of the barrier 126. In the gap part 136, auxiliary electrodes 137A and 137B may be protruded toward the surface of the barrier 126. Referring to FIG. 16, a first auxiliary electrode 137A protrudes from the 1-1 gap part 136A1 among the first gap parts 136A. The first auxiliary electrode 137A protrudes from a first stepped connection surface 136A1, (see FIG. 18) constituting the 1-1 gap part 136A1 toward the electrode connection surface 141B. The first auxiliary electrode 137A connects between the first stepped connection surface 136A1 and the first stepped surface 136A2 (see FIG. 18) of the first electrode part 130A. The first auxiliary electrodes 137A and 137B may be seen as parts that induce polarization by bringing the first gap part 136A and the surface of the barrier 126 closer together.

The first electrode 130A has first surface 132A and second surface 133A and a first electrode side surface 135A′,136A2 between the first and second surfaces 132A, 133A. The second electrode 130B is disposed to be spaced apart from the first electrode 130A and has first surface 132B and second surface 133B and a second electrode side surface 135B′,136B2 between the first and second surfaces 132B, 133B. The barrier 126 made of a dielectric material disposed between the first electrode side surface 135A′,136A2 and the second electrode side surface 135B′,136B2. And, the ultraviolet (UV) lamp 140 has the first surface 141B and the second surface 141A which are opposite surfaces. The first surface 141B of the UV lamp 140 covers over the first surface 132A of the first electrode 130A, the first surface 132B of the second electrode 130B, and the barrier 126, and the second surface of the UV lamp 140 has the light extraction surface 141A. The prescribed gas of the UV lamp 140 is configured for an excimer discharge of light through the light extraction surface 141A when the first and second electrodes 130A, 130B are configured to receive a voltage. At least one of the first electrode surface or the second electrode surface includes a stepped surface 136A2, 136B2 forming a step 136A, 136B. And a wall surface 126A1,126A2 of the barrier 126 is spaced apart from the stepped surface 136A2, 136B2.

In this embodiment, the first auxiliary electrode 137A has an approximately semicircular shape based on the plane. As another example, the first auxiliary electrode 137A may have a polygonal shape whose width gradually narrows toward the surface of the barrier 126. As another example, the first auxiliary electrode 137A may widen toward the surface of the barrier 126 or may have the same width.

For reference, the structure of the second electrode part 130B is symmetrical to the structure of the first electrode part 130A. As shown in FIG. 12, the second electrode part 130B has the second electrode protrusion 135B, and the second electrode protrusion 135B is composed of a 2-1 electrode protrusion 135B1 and a 2-2 electrode protrusion 135B2 arranged on both sides based on the second electrode groove (reference numeral not given). The 2-1 electrode protrusion 135B1 and the 2-2 electrode protrusion 135B2 may be provided with the 2-1 gap part 136B1 and the 2-2 gap part 136B2, respectively.

The second electrode part 130B may also be provided with a second auxiliary electrode 137B. As shown in FIG. 15, the second auxiliary electrode 137B is arranged in a complementary position to the first auxiliary electrode 137A. That is, the second auxiliary electrode 137B may be provided in the 2-2 gap part 136B2. The specific structure of the second electrode part 130B will be referred to the structure of the first electrode part 130A.

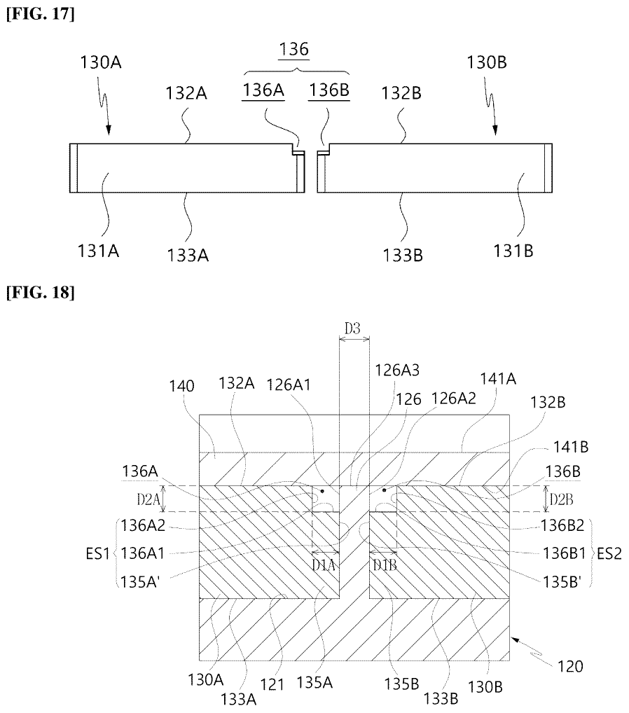

Referring to FIG. 17, a state in which the pair of electrode parts 130A, 130B face each other is illustrated. As may be seen, the first electrode part 130A and the second electrode part 130B are arranged to face each other, and the first gap part 136A and the second gap part 136B may also face each other. Of course, since the barrier 126 is arranged between them, the first gap part 136A and the second gap part 136B are not directly connected to each other.

FIG. 18 illustrates a cross-sectional view of the far ultraviolet light emitting device (C). First, looking at the first electrode part 130A, a first electrode surface ES1 is formed on the first electrode part 130A. The first electrode surface ES1 may include a first barrier facing surface 135A′, the first stepped surface 136A2, and the first stepped connection surface 136A1.

The first barrier facing surface 135A′ may face the surface 126A1 of the barrier 126 and be spaced apart from the surface 126A1 of the barrier 126 by a first distance. The first stepped surface 136A2 may face the surface 126A1 of the barrier 126 and be spaced apart from the surface 126A1 of the barrier 126 by a second distance longer than the first distance. The first stepped connection surface 136A1 may connect the first barrier facing surface 135A′ and the first stepped surface 136A2.

The first barrier facing surface 135A′ of the first electrode part 130A may be viewed as the surface of the first electrode protrusion 135A. In the present embodiment, the first barrier facing surface 135A′ of the first electrode part 130A is in close contact with the surface of the barrier 126, so that the first distance becomes 0. In another example, the first distance may be formed to be greater than 0, so that the first barrier facing surface 135A′ of the first electrode part 130A and the surface of the barrier 126 may be spaced apart from each other.

The first stepped surface 136A2 may form a step between the first electrode part 130A and the barrier 126. Here, the step means that the separation distance between the first electrode part 130A and the barrier 126 increases so as to be separated between the first electrode part 130A and the barrier 126.

Since the first stepped surface 136A2 of the first electrode part 130A is spaced apart from the surface 126A1 of the barrier 126, the first gap part 136A may be formed between the first stepped surface 136A2 of the first electrode part 130A and the surface 126A1 of the barrier 126. Here, a separate direction between the first stepped surface 136A2 of the first electrode part 130A and the surface 126A1 of the barrier 126 is a direction in which the first electrode part 130A and the second electrode part 130B are spaced apart from each other, that is, a left-right direction based on FIG. 18.

The first stepped connection surface 136A1 may be formed between the first barrier facing surface 135A′ of the first electrode part 130A and the first stepped surface 136A2 of the first electrode part 130A. The first stepped connection surface 136A1 connects the first barrier facing surface 135A′ of the first electrode part 130A and the first stepped surface 136A2 of the first electrode part 130A. The first stepped connection surface 136A1 may face the electrode connection surface 141B. The first stepped connection surface 136A1 may have a length (D1A) equal to the second distance.

Referring to FIG. 18, the first gap part 136A may be viewed as an empty space surrounded by (i) the first stepped surface 136A2 of the first electrode part 130A, (ii) the first stepped connection surface 136A1, (iii) the first barrier surface 126A1 of the barrier 126, and (iv) the electrode connection surface 141B of the ultraviolet lamp 140. The first gap part 136A has a structure that opens toward the electrode connection surface 141B. The first stepped surface 136A2 of the first electrode part 130A may be arranged closer to the electrode connection surface 141B than the first barrier facing surface 135A′ of the first electrode part 130A.

Similarly, at the second electrode part 130B, a second electrode surface ES2 is formed on the second electrode part 130B. The second electrode surface ES2 may include a second barrier facing surface 135B′, a second stepped surface 136B2, and a second stepped connection surface 136A1. The second barrier facing surface 135B′ may face the surface 126A2 of the barrier 126 and be spaced apart from the surface 126A2 of the barrier 126 by a first distance. The second stepped surface 136B2 may face the surface 126A2 of the barrier 126 and be spaced apart from the surface 126A2 of the barrier 126 by a second distance longer than the first distance. The second stepped connection surface 136B1 may connect the second barrier facing surface 135B′ and the second stepped surface 136B2.

The second stepped surface 136B2 may form a step between the second electrode part 130B and the barrier 126. Here, the step means that the separation distance between the second electrode part 130B and the barrier 126 increases so as to be separated between the second electrode part 130B and the barrier 126.

The second stepped connection surface 136B1 connects the second barrier facing surface 135B′ of the second electrode part 130B and the second stepped surface 136B2 of the second electrode part 130B. The second stepped connection surface 136B1 may face the electrode connection surface 141B. The second stepped connection surface 136B1 may have a length (D1B) equal to the second distance.

Referring to FIG. 18, the second gap part 136B may be viewed as an empty space surrounded by (i) the second stepped surface 136B2 of the second electrode part 130B, (ii) the second stepped connection surface 136B1, (iii) the second barrier surface 126A2 of the barrier 126, and (iv) the electrode connection surface 141B of the ultraviolet lamp 140. The second gap part 136B has a structure that opens toward the electrode connection surface 141B. The second stepped surface 136B2 of the second electrode part 130B may be arranged closer to the electrode connection surface 141B than the second barrier facing surface 135B′ of the second electrode part 130B.

It is preferable that a separation distance D1A between the first stepped surface 136A2 of the first electrode part 130A and the surface 126A1 of the barrier 126 is between 0.8 to 1.2 times a separation distance D2A between the first stepped connection surface 136A1 and the electrode connection surface 141B. That is, the ratio D1A/D2A of the two separations is preferably in the range of 0.8 to 1.2.

This is because when the separation distance D1A between the first stepped surface 136A2 of the first electrode part 130A and the surface 126A1 of the barrier 126 is relatively excessively short, the probability that a spark occurring between the first electrode part 130A and the second electrode part 130B is increased. On the contrary, when the separation distance D1A between the first stepped surface 136A2 of the first electrode part 130A and the surface 126A1 of the barrier 126 is relatively excessively wide, the discharge electric field in the first gap part 136A is weakened, thereby increasing the probability that the ultraviolet lamp 140 is not turned on.

Table 1 below summarizes the results of testing the lighting probability of the ultraviolet lamp 140 in the present embodiment in which the ratio (D1A/D2A) of the two separation distances is variously applied, and the results using the conventional technology as a comparative example. For reference, the applied voltage is set to 6 kV, and the comparative example is a structure without the barrier 126, that is, a structure in which the pair of electrode parts are spaced apart, but there are no obstacles between the electrode parts. And the comparative example is tested while varying the distance between the pair of electrode parts, and the comparative example is tested for lighting without a separate auxiliary electrode or auxiliary light source.

TABLE 1

| | |
|---|---|
| Present embodiment | comparative example |

| | | Lighting | Distance between | Lighting |
|---|---|---|---|---|
| | | probability | electrode | probability |
| | D1A/D2A | (%) | parts(mm) | (%) |
| 1 | 0.5 | 99.2 | 8.0 | 28.1 |
| 2 | 0.6 | 99.4 | 9.0 | 29.4 |
| 3 | 0.7 | 98.9 | 10.0 | 27.9 |
| 4 | 0.8 | 99.5 | 11.0 | 20.3 |
| 5 | 0.9 | 99.1 | 12.0 | 22.1 |
| 6 | 1.0 | 98.7 | 13.0 | 17.5 |
| 7 | 1.1 | 98.8 | 14.0 | 15.6 |
| 8 | 1.2 | 98.5 | 15.0 | 16.8 |
| 9 | 1.3 | 89.6 | 16.0 | 10.4 |
| 10 | 1.4 | 85.4 | 17.0 | 9.3 |

As shown in Table 1 above, according to the embodiment of the present disclosure, it may be seen that when the ratio (D1A/D2A) of the two separation distances is 1.2 or less, the ultraviolet lamp 140 is turned on with a very high probability. On the other hand, in the case of the comparative example, since there is no separate auxiliary lighting means, the lighting probability is very low even if the distance between the electrode parts is close.

Table 2 below summarizes the results of testing the probability of a spark occurring between the pair of electrode parts 130A and 130B in the embodiment in which the ratio (D1A/D2A) of the two separation distances above is variously applied, and the results using the conventional technology as a comparative example. For reference, the applied voltage is set to 6 kV, and the comparative example is a structure without the barrier above 126, that is, a structure in which the pair of electrode parts are spaced apart, but there are no obstacles between the electrode parts. And the comparative example is tested while varying the distance between the pair of electrode parts.

TABLE 2

| | |
|---|---|
| Present embodiment | comparative example |

| | | Spark | Distance between | Spark |
|---|---|---|---|---|
| | | probability | electrode | probability |
| | D1A/D2A | (%) | parts(mm) | (%) |
| 1 | 0.5 | 37.1 | 8.0 | 85.0 |
| 2 | 0.6 | 29.5 | 9.0 | 74.5 |
| 3 | 0.7 | 16.5 | 10.0 | 70.8 |
| 4 | 0.8 | 8.1 | 11.0 | 55.8 |
| 5 | 0.9 | 6.5 | 12.0 | 52.0 |
| 6 | 1.0 | 6.7 | 13.0 | 42.8 |
| 7 | 1.1 | 5.1 | 14.0 | 38.3 |
| 8 | 1.2 | 4.8 | 15.0 | 29.8 |
| 9 | 1.3 | 4.1 | 16.0 | 19.6 |
| 10 | 1.4 | 3.6 | 17.0 | 18.9 |

As shown in Table 2 above, according to the embodiment of the present disclosure, when the ratio (D1A/D2A) of the two separation distances is 0.8 or more, it may be seen that the probability of a spark occurring between the pair of electrode parts 130A and 130B is very low. On the other hand, in the case of the comparative example, since there is no separate barrier 126 between the pair of electrode parts, there is a high probability that a spark occurs even if the distance between the electrode parts is far. As a result, the ratio (D1A/D2A) of the two separation distances is preferably 0.8 to 1.2.

Meanwhile, the separation distance D1A between the first stepped surface 136A2 of the first electrode part 130A and the first barrier surface 126A1 of the barrier 126 may be formed to be greater than or equal to the thickness D3 of the barrier 126 based on a direction in which the first electrode part 130A and the second electrode part 130B are spaced apart from each other. This is because when the thickness D3 of the barrier 126 is relatively thin, the probability of a spark occurring between the pair of electrode parts 130A and 130B becomes very high.

Referring to FIG. 18, the maximum separation distance D1A between the surface 136A2 of the first electrode part 130A forming the first gap part 136A and the surface 126A1 of the barrier 126 is preferably between 0.8 to 1.2 times a maximum separation distance D2A between the surface 136A1 of the first electrode part 130A forming the gap part 136 and the electrode connection surface 141B. Here, the maximum separation distance D1A between the surface 136A2 of the first electrode part 130A forming the first gap part 136A and the surface 126A1 of the barrier 126 means the maximum separation distance between the first stepped surface 136A2 of the first electrode part 130A and the first barrier surface 126A1. And the maximum separation distance D2A between the surface 136A1 of the first electrode part 130A forming the gap part 136 and the electrode connection surface 141B means the maximum separation distance between the first stepped connection surface 136A1 of the first electrode part 130A and the electrode connection surface 141B. In this embodiment, since the first stepped surface 136A2 of the first electrode part 130A and the first stepped connection surface 136A1 of the first electrode part 130A are each flat, the two maximum separation distances D1A and D2A are the same in all planes, but if they are not flat, the spaced distance may vary depending on the position.

Meanwhile, since the ultraviolet lamp 140 is disposed to cross the first electrode part 130A, the barrier 126, and the second electrode part 130B, the electrode connection surface 141B may face the upper surface 132A of the first electrode part 130A, the upper surface of the barrier 126, and the upper surface 132B of the second electrode part 130B, respectively. In this case, the electrode connection surface 141B may be in contact with the upper surface 132A of the first electrode part 130A, the upper surface of the barrier 126, and the upper surface 132B of the second electrode part 130B, respectively.

In this case, the fact that the ultraviolet lamp 140 is disposed across the first electrode part 130A, the barrier 126, and the second electrode part 130B means that the same surface (here, the electrode connection surface 141B) of the ultraviolet lamp 140 is disposed to face the first electrode part 130A, the barrier 126, and the second electrode part 130B, respectively. That is, among a plurality of surfaces constituting the ultraviolet lamp 140, the same surface faces the first electrode part 130A, the barrier 126, and the second electrode part 130B, respectively.

In the present embodiment, the light extraction surface 141A of the surface of the ultraviolet lamp 140 is disposed to face opposite sides of the first electrode part 130A, the barrier 126, and the second electrode part 130B. The electrode connection surface 141B, which is the opposite surface of the light extraction surface 141A, is disposed to face the first electrode part 130A, the barrier 126, and the second electrode part 130B.

In this way, when the ultraviolet lamp 140 is disposed across the first electrode part 130A, the barrier 126, and the second electrode part 130B, surface discharge may be performed in the ultraviolet lamp 140. The surface discharge may induce a stable discharge of the far ultraviolet light emitting device (C) along with the start of the ultraviolet lamp 140 through the concentration of the discharge electric field around the gap part 136.

In the present embodiment, each of the electrode connection surface 141B and the light extraction surface 141A of the ultraviolet lamp 140 has a planar shape. The electrode connection surface 141B is disposed across the first electrode part 130A, the barrier 126, and the second electrode part 130B, and the light extraction surface 141A faces a direction in which far UVC rays are irradiated. For example, the light extraction surface 141A may face the interior space. In this case, since the electrode connection surface 141B is in charge of contact with the first electrode part 130A, the barrier 126, the second electrode part 130B, and the like, the components interfering with the light extraction surface 141A may be minimized. Accordingly, the light extraction surface 141A may focus entirely on irradiating far UVC rays.

The total length of the ultraviolet lamp 140 may be longer than the sum of lengths of the first electrode part 130A, the barrier 126, and the second electrode part 130B. Accordingly, both end portions of the ultraviolet lamp 140 may protrude to the outside of the first electrode part 130A and the second electrode part 130B, respectively. As another example, the total length of the ultraviolet lamp 140 may be shorter than or equal to the sum of lengths of the first electrode part 130A, the barrier 126, and the second electrode part 130B.

The ultraviolet lamp 140 may be an excimer lamp. The excimer refers to a dimer molecule formed of one atom in a ground state having the lowest energy and one atom in an excited state having a certain level of high energy. The ultraviolet lamp 140 may be composed of a lamp filled with an appropriate excimer emission gas in a discharge container made of quartz glass. Accordingly, light (ultraviolet light) may be irradiated when the excimer transitions to the ground state.

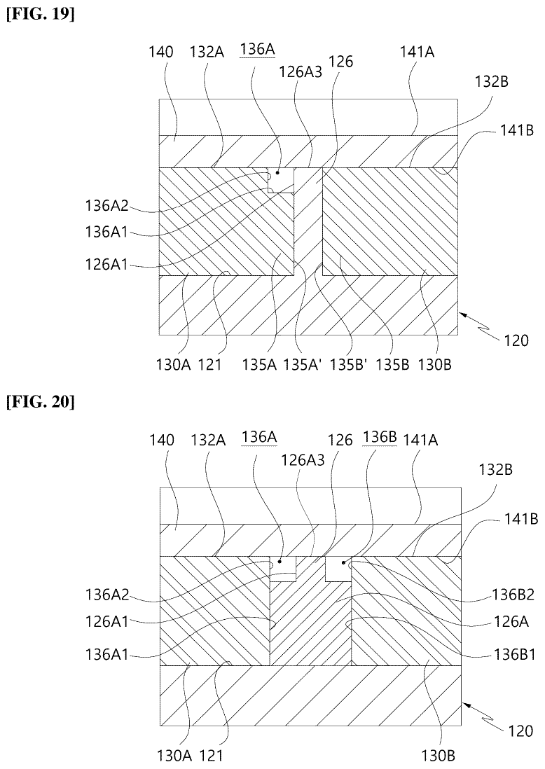

In FIGS. 19 to 25, various modified embodiments of the pair of electrode parts 130A and 130B and the barrier 126 constituting the present disclosure are illustrated in a cross-sectional view. Hereinafter, the same structure as the previous embodiment will be omitted, and only a structure different from the previous embodiment will be described.

First, referring to FIG. 19, among the pair of electrode parts 130A and 130B, the gap part 136 may be provided only between the first electrode part 130A and the barrier 126. That is, the gap part 136 is omitted between the second electrode part 130B and the barrier 126. The gap part 136 becomes the first gap part 136A, and the first gap part 136A may be formed by being surrounded by the first stepped surface 136A2 of the first electrode part 130A, the first barrier surface 126A1 of the barrier 126, and the electrode connection surface 141B of the ultraviolet lamp 140.

Referring to an embodiment shown in FIG. 20, the barrier 126 is configured separately from the lamp housing 120. The barrier 126 may be mounted on the lamp housing 120 to barrier the mounting space 122. The barrier 126 may be composed of two parts having different thicknesses. Here, the thickness is based on a left-right direction with respect to FIG. 20, that is, a direction in which the first electrode part 130A and the second electrode part 130B are spaced apart from each other.

More specifically, the barrier 126 may include a first barrier portion and a second barrier portion having different thicknesses. Although not classified by different reference numerals, the first barrier portion is provided relatively far from the electrode connection portion and serves as a base portion of the barrier 126. The second barrier portion is connected to the first barrier portion and is disposed closer to the electrode connection surface 141B than the first barrier portion.

In this case, the thickness of the second barrier portion based on a direction in which the first electrode part 130A and the second electrode part 130B are spaced apart from each other is thinner than that of the first barrier portion, and the gap part 136A, 136E may be formed between the surfaces 126A1 and 126A2 of the second barrier portion and the surfaces 136A2 and 136B2 of the first electrode part 130A or the second electrode part 130B. That is, in the present embodiment, the gap part 136A, 136E may be formed in a thin portion of the barrier 126 rather than by a structure recessed in a portion of the pair of electrode parts 130A and 130B. In another embodiment, the barrier 126 may be integrally provided with the lamp housing 120.

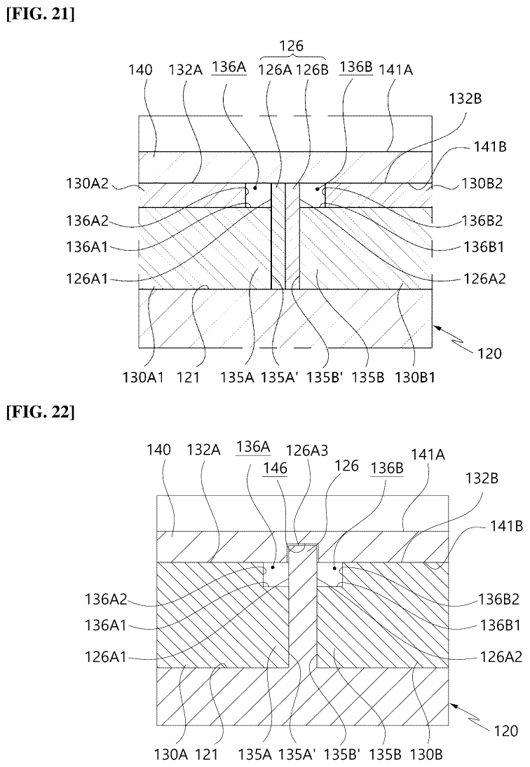

According to an embodiment shown in FIG. 21, the first electrode part 130A is formed by stacking two different electrode bodies 130A1 and 130A2. More specifically, the first electrode part 130A of the pair of electrode parts 130A and 130B includes a 1-1 electrode body 130A1 spaced apart from the barrier 126 by a first distance and a 1-2 electrode body 130A2 spaced apart from the barrier 126 by a second distance longer than the first distance and disposed between the first body 130A1 and the ultraviolet lamp 140. In this case, the 1-1 electrode body 130A1 receives power, and the 1-2 electrode body 130A2 is in contact with the ultraviolet lamp 140.

One of the two electrode bodies 130A1 and 130A2 may be spaced apart from the surface 126A1 of the barrier 126 to form the gap part 136A, 136E. The first electrode part 130A includes the 1-1 electrode body 130A1 and a the 1-2 electrode body 130A2. The 1-2 electrode body 130A2 may be shorter than the 1-1 electrode body 130A1, and a stepped surface 136A2 may be formed between the 1-1 electrode body 130A1 and the 1-2 electrode body 130A2, and the stepped surface 136A2 may form the first gap part 136A between the first barrier surface 126A1 of the barrier 126.

Likewise, the second electrode part 130B is formed by stacking two different electrode bodies 130B1 and 130B2. One of the two electrode bodies 130B1 and 130B2 may be spaced apart from the surface of the barrier 126 to form the gap part 136E. More specifically, the second electrode part 130B includes a 2-1 electrode body 130B1 and a 2-2 electrode body 130B2. The 2-2 electrode body 130B2 may be provided shorter than the 2-1 electrode body 130B2, and a stepped surface 136B2 may be formed between the 2-1 electrode body 130B1 and the 2-2 electrode body 130B2, and the stepped surface 136B2 may form a second gap part 136B between the first barrier surface 126A1 and the barrier 126.

In the present embodiment, the barrier 126 is configured separately from the lamp housing 120. The barrier 126 includes a plurality of components. The barrier 126 includes a first barrier body 126A and a second barrier body 126B. The first barrier body 126A and the second barrier body 126B may be stacked in a direction in which the first electrode part 130A and the second electrode part 130B are spaced apart from each other, that is, in a left-right direction based on the drawing, to constitute one barrier 126. The entire thickness of the barrier 126 composed of a plurality of components may be varied through component replacement or component omission.

According to an embodiment illustrated in FIG. 22, the upper surface of the barrier 126 facing the electrode connection surface 141B may protrude further toward the electrode connection surface 141B than the upper surface 132A of the first electrode part 130A and the upper surface 132B of the second electrode part 130B. A coupling groove 146 into which the upper portion of the barrier 126 is inserted is formed on the electrode connection surface 141B of the ultraviolet lamp 140. Accordingly, the upper portion of the barrier 126 protruding further toward the electrode connection surface 141B than the upper surface 132A of the first electrode part 130A and the upper surface 132B of the second electrode part 130B may be inserted into the coupling groove 146. In this case, the contact area between the barrier 126 and the ultraviolet lamp 140 is increased, thereby increasing electrical polarization efficiency

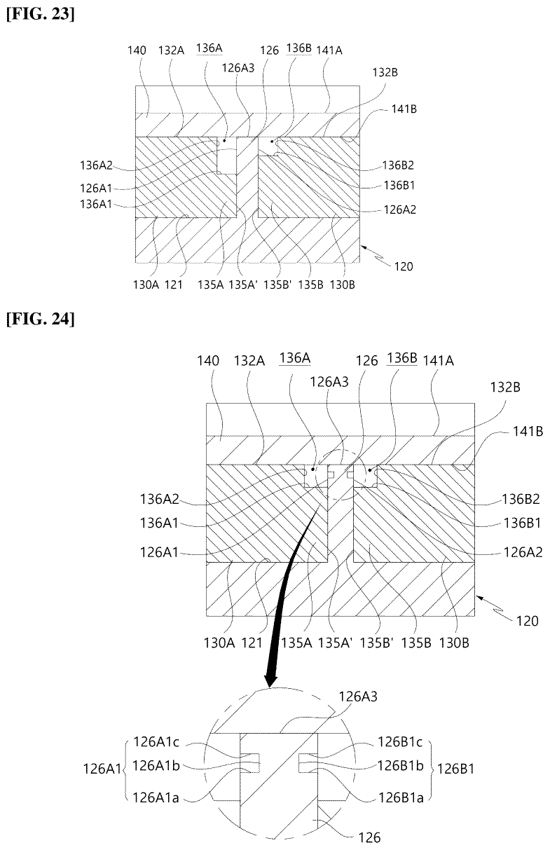

According to an embodiment shown in FIG. 23, the first gap part 136A and the second gap part 136B have asymmetrical shapes. More specifically, the vertical length of the first stepped surface 136A2 of the first electrode part 130A is longer than the vertical length of the second stepped surface 136B2 of the second electrode part 130B. Accordingly, the first stepped connection surface 136A1 is spaced apart from the electrode connection surface 141B of the ultraviolet lamp 140 more than the second stepped connection surface 136B1.

According to an embodiment shown in FIG. 24, the first barrier surface 126A1 facing the surface of the first electrode part 130A and the second barrier surface 126A2 facing the surface of the second electrode part 130B are formed in the barrier 126. In this case, barrier recess 126A1′, 126B1′ are formed in at least one of the first barrier surface 126A1 and the second barrier surface 126A2 in a direction opposite to a direction in which the first electrode part 130A and the second electrode part 130B are spaced apart from each other. In this embodiment, a first barrier recess 126A1′ is formed on the first barrier surface 126A1, and a second barrier recess 126B1′ is formed on the second barrier surface 126A2.

Referring to the first barrier recess 126A1′, the first barrier recess 126A1′ includes a first recess lower surface 126A1a, a first recess side surface 126A1b, and a first recess upper surface 126A1c. It may be viewed that the first barrier recess 126A′ is recessed in a direction facing the first gap part 136A. That is, the first barrier recess 126A1′ may increase the width of the first gap part 136A, more specifically, the width based on the direction in which the pair of electrode parts 130A and 130B are spaced apart from each other.

Referring to the second barrier recess 126B1′, the second barrier recess 126B1′ includes a second recess lower surface 126B1a, a second recess side surface 126B1b, and a second recess upper surface 126B1c. The second barrier recess 126B1′ may be recessed in a direction facing the second gap part 136B. That is, the second recess 126B1′ may increase the width of the second gap part 136B, more precisely, the width based on the direction in which the pair of electrode parts 130A and 130B are spaced apart from each other. As a result, the gap part 136 includes portions having different distances between the surfaces 136A2 and 136B2 of the electrode parts 130A and 130B and the surfaces 126A1 and 126A2 of the barrier 126.

As described above, when the first barrier recess 126A1′ and the second barrier recess 126B1′ are provided, even if a spark occurs between the pair of electrode parts 130A and 130B, a point of spark occurring may be guided to the inside of the first barrier recess 126A1′ and/or the second barrier recess 126B1′. Accordingly, stability may be increased when the far ultraviolet light emitting device (C) is operated. Also, the surface area of the widened gap parts 136A and 136B may further increase the electric field concentration to help the ultraviolet lamp 140 to be turned on.

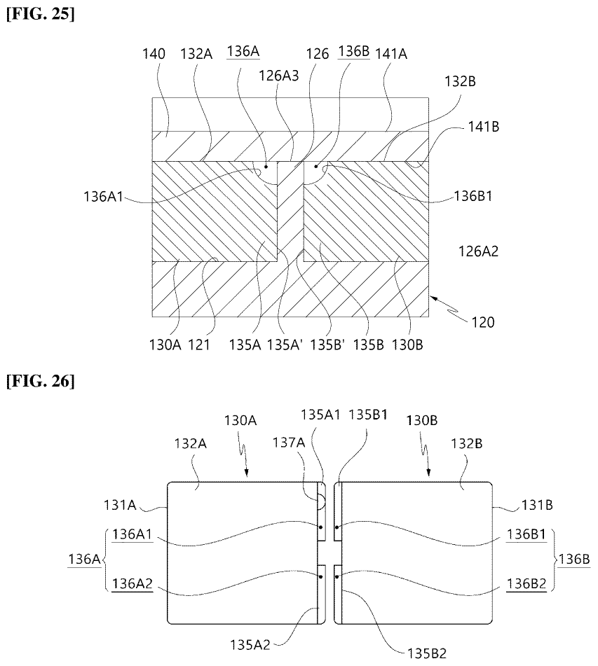

According to an embodiment shown in FIG. 25, the surfaces 136A1 and 136B1 of the gap parts 136A and 136B are formed to be inclined or curved such that the width opened from the gap parts 136A and 136B toward the electrode connection surfaces 141B is gradually widened. More specifically, the surface 136B1 of the first gap part 136A and the surface 136B1 of the second gap part 136B are curved, respectively. The first gap part 136A and the second gap part 136B may be formed to face the barrier 126 and the electrode connection surface 141B. In this case, the widths of the first gap part 136A and the second gap part 136B are gradually widened toward the electrode connection surface 141B.

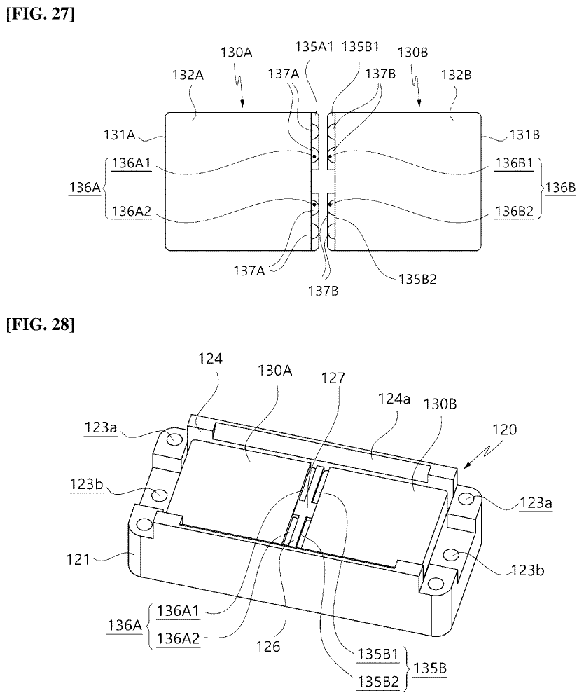

In FIGS. 26 and 27, other embodiments of the auxiliary electrode 137A provided in the pair of electrode parts 130A and 130B constituting the present disclosure are illustrated. First, referring to FIG. 26, the auxiliary electrode 137A may be provided on the first electrode part 130A, and the auxiliary electrode may be omitted from the second electrode part 130B. Referring to an embodiment illustrated in FIG. 27, the auxiliary electrodes 137A and 137B may be provided on the first electrode part 130A and the second electrode part 130B, respectively. In this case, a plurality of auxiliary electrodes 137A and 137B may be provided on the first electrode part 130A and the second electrode part 130B, respectively.

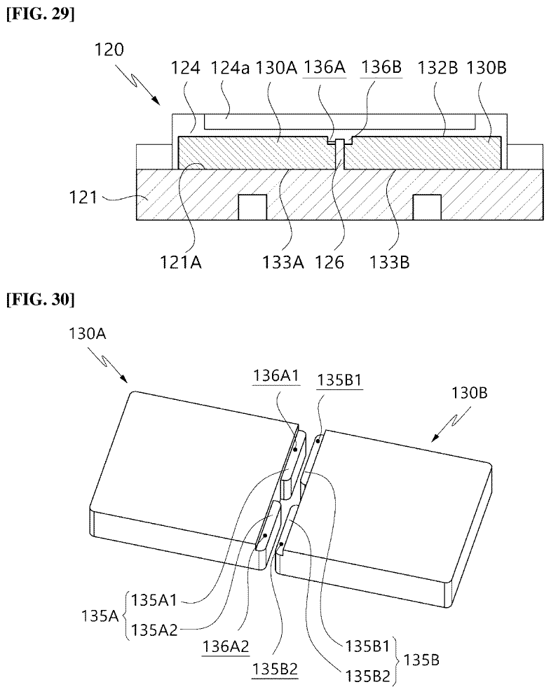

In an embodiment illustrated in FIGS. 28 to 30, a separate auxiliary electrode may be omitted from the pair of electrode parts 130A and 130B. The first electrode part 130A and the second electrode part 130B constituting the pair of electrode parts 130A and 130B are disposed to face each other, and the electrode protrusions 135A and 135B are provided on surfaces of the first electrode part 130A and the second electrode part 130B facing each other. The first electrode part 130A is provided with the 1-1 electrode protrusion 135A1 and the 1-2 electrode protrusion 135A2 spaced apart from each other, and the second electrode part 130B is provided with the 2-1 electrode protrusion 135B1 and the 2-2 electrode protrusion 135B2 spaced apart from each other.

The first barrier surface 126A1 facing a surface of the first electrode part 130A and the second barrier surface 126A2 facing a surface of the second electrode part 130B are formed in the barrier 126. In this case, a thickness D3 (see FIG. 18), which is a distance between the first barrier surface 126A1 and the second barrier surface 126A2, may be proportional to a magnitude of a voltage applied to the first electrode part 130A and the second electrode part 130B.

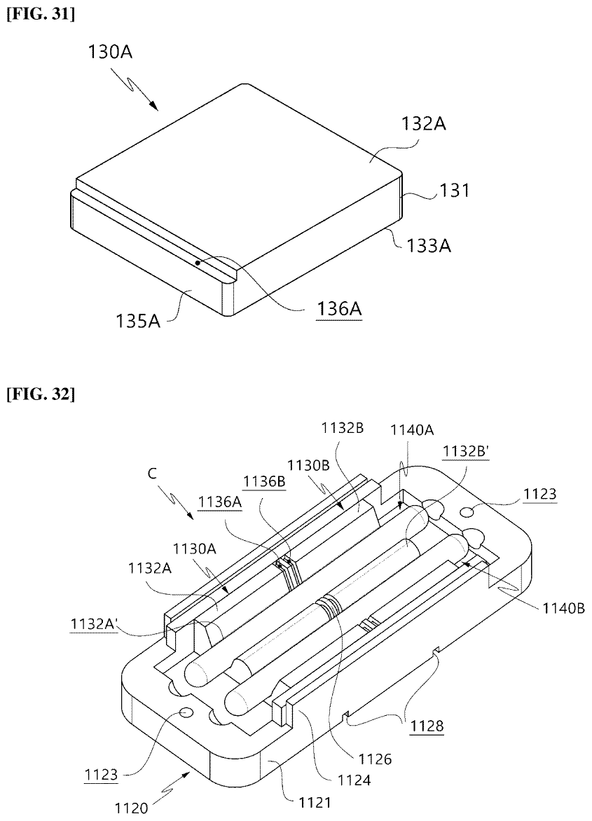

According to an embodiment illustrated in FIG. 31, the first electrode part 130A may be provided with one first electrode protrusion 135A connected continuously to each other, and the first gap part 136A formed by the first electrode protrusion 135A may also have a connected structure. Although only the first electrode part 130A is illustrated in the drawing, the second electrode part 130B spaced apart from the first electrode part 130A may also have the same structure as the first electrode part 130A.

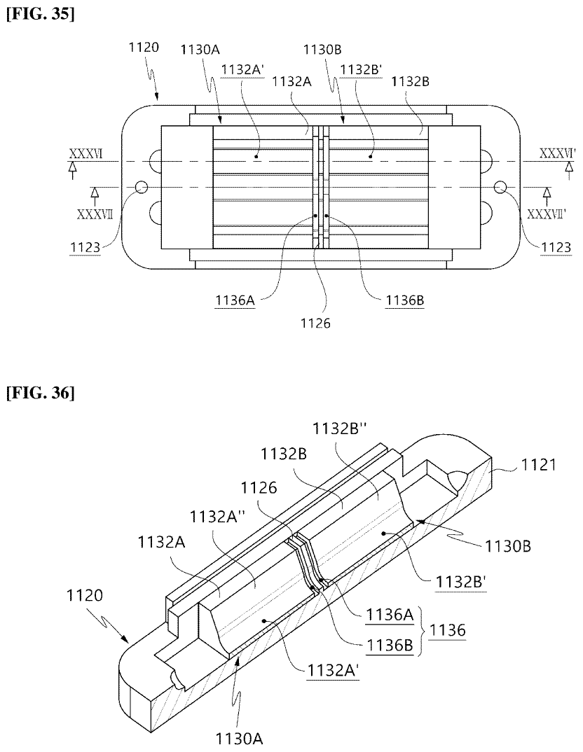

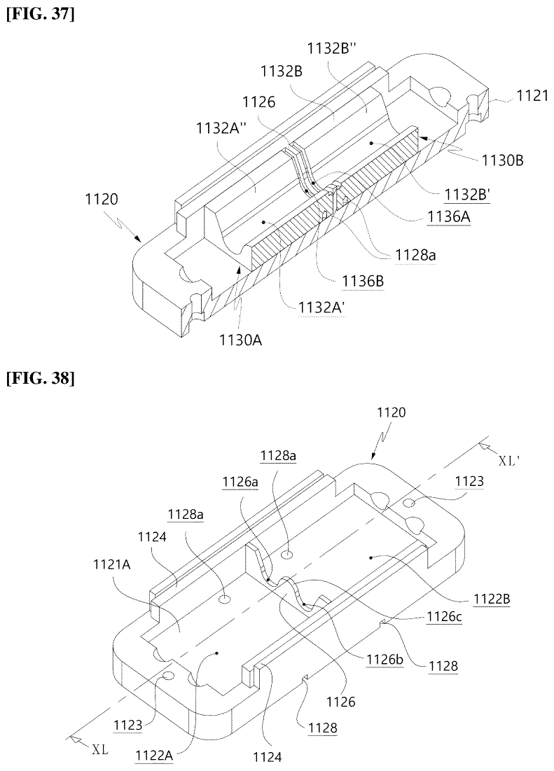

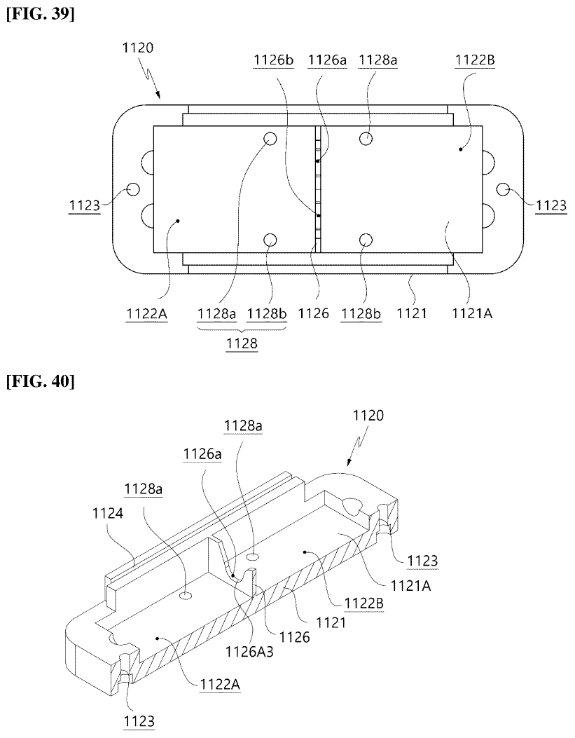

In FIGS. 32 to 46, another embodiment of the far ultraviolet light emitting device (C) according to the present disclosure is illustrated. The same structure as the above-described embodiments will be omitted, and other structures will be mainly described.

As illustrated in FIG. 32, a pair of electrode parts 1130A and 1130B are disposed in the lamp housing 1120 in the far ultraviolet light emitting device (C), and the barrier 1126 is disposed between the pair of electrode parts 1130A and 1130B. The barrier 1126 has a structure that blocks the first electrode part 1130A and the second electrode part 1130B constituting the pair of electrode parts 1130A and 1130B. The barrier 1126 is disposed across the lamp housing 1120, and in FIG. 32, the ultraviolet lamps 1140A and 1140B are disposed above the barrier 1126 so that a portion of the barrier 1126 is covered.

In this embodiment, the ultraviolet lamps 1140A and 1140B have a tube structure. More precisely, the surfaces of the ultraviolet lamps 1140A and 1140B have a curved shape, and seating grooves 1132A′ and 1132B′ having a curved shape, in which the surfaces of the ultraviolet lamps 1140A and 1140B are seated, are formed in the first electrode part 1130A and the second electrode part 1130B. As shown in FIG. 32, the ultraviolet lamps 1140A and 1140B may have a tube structure having an approximately circular cross section. That is, the ultraviolet lamps 1140A and 1140B have an approximately cylindrical shape. As another example, the cross-sections of the ultraviolet lamps 1140A and 1140B may have an oval shape or a shape in which a curve and a straight line are mixed.

In this embodiment, the ultraviolet lamps 1140A and 1140B include a first ultraviolet lamp 1140A and a second ultraviolet lamp 1140B. The two ultraviolet lamps 1140A and 1140B may be disposed in parallel to the far ultraviolet light emitting device (C). The two ultraviolet lamps 1140A and 1140B may be disposed at two seating portions to be described later, respectively.

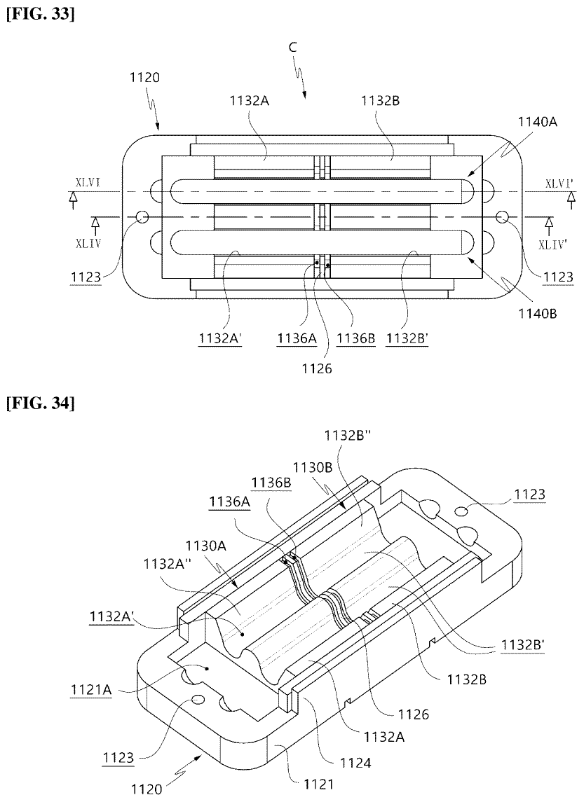

Referring to FIG. 33, the ultraviolet lamps 1140A and 1140B are disposed across the first electrode part 1130A, the barrier 1126, and the second electrode part 1130B. The ultraviolet lamps 1140A and 1140B are disposed on the upper portion of the first electrode part 1130A, the upper portion of the barrier 1126, and the upper portion of the second electrode part 1130B. The total length of the ultraviolet lamps 1140A and 1140B may be greater than the sum of the lengths of the first electrode part 1130A, the barrier 1126, and the second electrode part 1130B.

Referring to FIG. 34, an appearance of the far ultraviolet light emitting device (C), from which the ultraviolet lamps 1140A and 1140B are omitted, is illustrated. As may be seen, consecutive seating grooves 1132A′ and 1132B′ may be formed in the first electrode part 1130A and the second electrode part 1130B. More specifically, two first seating grooves 1132A′ of the first electrode part 1130A are connected to two second seating grooves 1132B′ of the second electrode part 1130B, respectively. Reference numeral 1132A″ denotes a sidewall which forms the first seating grooves 1132A′, and reference numeral 1132B″ denotes a sidewall which forms the second seating grooves 1132B′.

In this case, referring to FIG. 38, the connecting grooves 1126a and 1126b are formed in the barrier 1126. The connecting grooves 1126a and 1126b are recessed in the barrier 1126 to have a curved shape in which the surfaces of the ultraviolet lamps 1140A and 1140B are seated. The seating grooves 1132A′ of the first electrode part 1130A, the connecting grooves 1126a and 1126b, and the seating grooves 1132B′ of the second electrode part 1130B form continuous seating portions. Referring to FIGS. 38 to 40, the connecting grooves 1126a and 1126b are composed of two adjacent connecting grooves 1126a and 1126b.

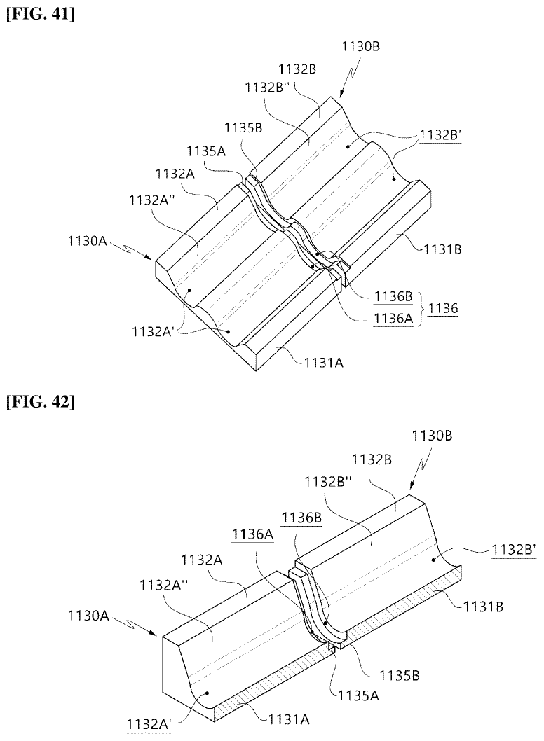

In this embodiment, the first gap part 1136A is provided between the first electrode part 1130A and the barrier 1126. The second gap part 1136B is provided between the second electrode part 1130B and the barrier 1126. Referring to FIGS. 41 to 43, the first gap part 1136A is formed by the first electrode protrusion 1135A of the first electrode part. The second gap part 1136B is formed by the second electrode protrusion 1135B of the second electrode part 1130B.

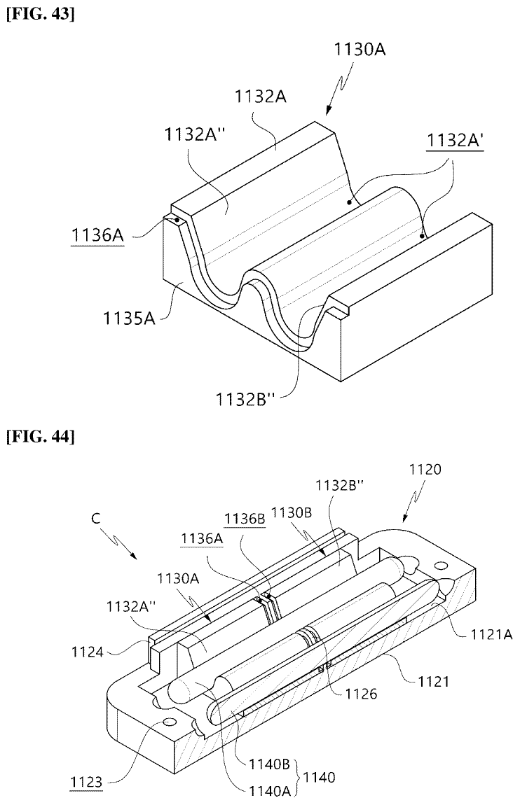

The first gap part 1136A is formed continuously along the surface of the first electrode part 1130A. Referring to FIG. 43, the upper surface of the first electrode protrusion 1135A is substantially a continuous uneven shape. More specifically, the upper surface of the first electrode protrusion 1135A may be a continuous curved surface, and thus may have a kind of wave pattern. Since the upper surfaces of the first electrode protrusion 1135A are continuously connected, the first gap part 1136A is also continuously formed without being disconnected. Since the second gap part 1136B has the same structure as the first gap part 1136A, a detailed description thereof will be omitted.

Referring to FIGS. 44 to 46, the embodiment is shown in a cross-sectional view. As may be seen, the first gap part 1136A and the second gap part 1136B are each provided continuously along the barrier 1126, and the first gap part 1136A and the second gap part 1136B have the lowest height on the lower portion of the ultraviolet lamp 1140A. That is, the first gap part 1136A and the second gap part 1136B are formed closest to the bottom surface of the lamp housing 1120, respectively, at portions facing the surface of the ultraviolet lamp 1140A.

Referring to FIG. 44, a portion of the first gap part 1136A and the second gap part 1136B is covered by the surface of the ultraviolet lamp 1140A, but the remaining portions of the first gap part 1136A and the second gap part 1136B that are positioned outside the ultraviolet lamp 1140A are exposed without being covered by the ultraviolet lamp 1140A. Since the remaining portions of the first gap part 1136A and the second gap part 1136B that are positioned outside the ultraviolet lamp 1140A are not in contact with the surface of the ultraviolet lamp 1140A, the influence on the start of the ultraviolet lamp 1140A, that is, the initial lighting is less affected than other portions. However, the remaining portions of the first gap part 1136A and the second gap part 1136B that are positioned outside the ultraviolet lamp 1140A may prevent sparks between the first electrode part 1130A and the second electrode part 1130B. In some cases, only a portion of the first gap part 1136A and the second gap part 1136B that are covered by the surface of the ultraviolet lamp 1140A may be viewed as the first gap part 1136A and the second gap part 1136B.

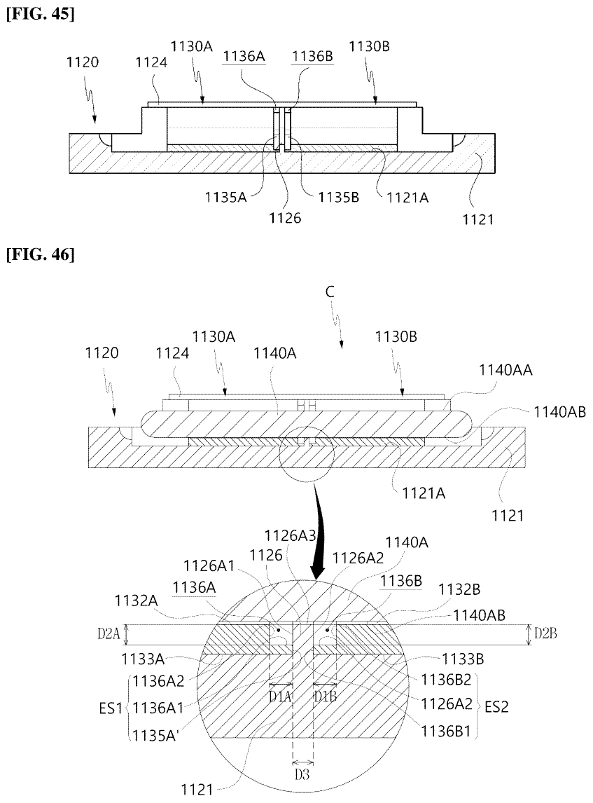

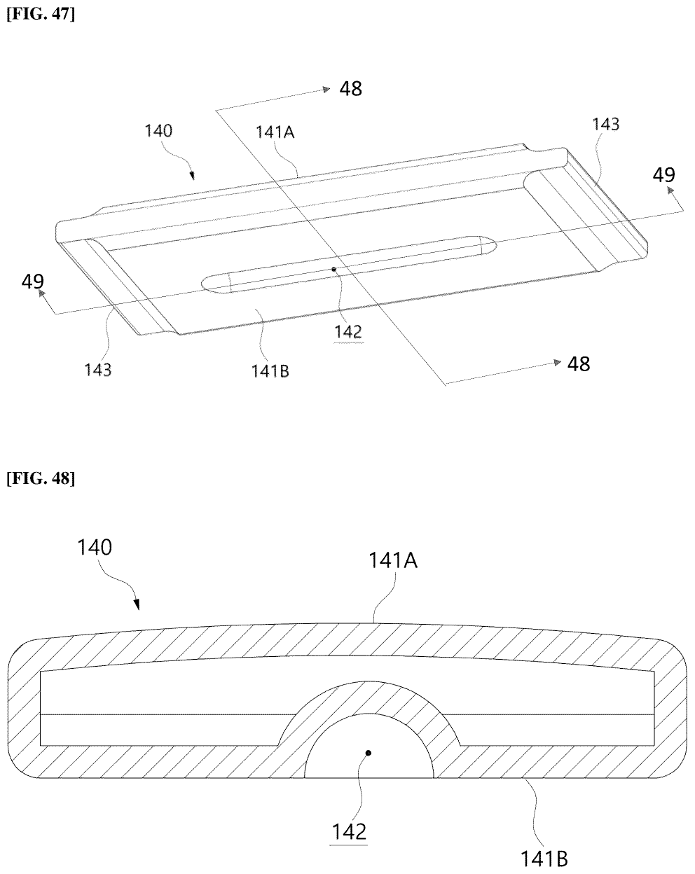

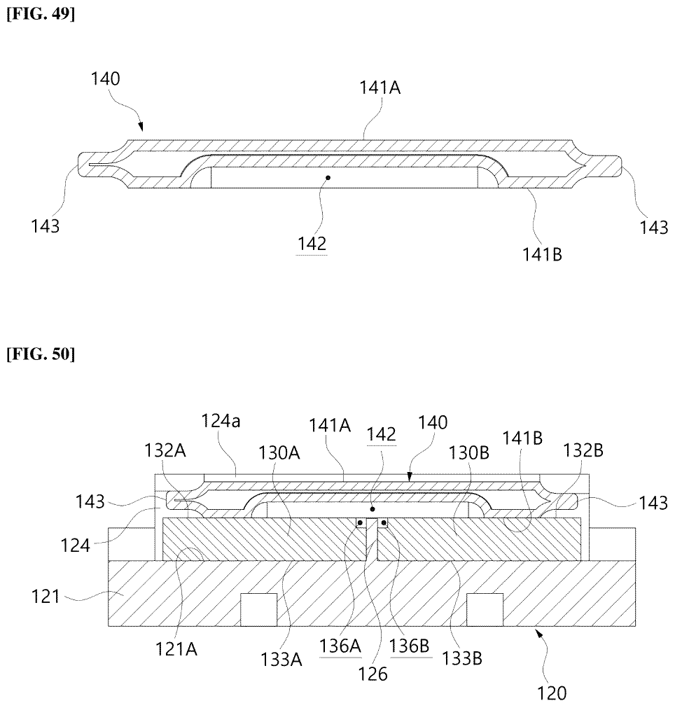

Referring to FIG. 45, it may be seen that the first gap part 1136A and the second gap part 1136B are formed from the top to the bottom of the barrier 1126 along the height direction (vertical direction based on FIG. 45). Among them, the ultraviolet lamps 1140A and 1140B may cross the lower portion of the barrier 1126, that is, the first gap part 1136A and the second gap part 1136B located at the lowest position.

Referring to FIG. 46, a state in which the ultraviolet lamps 1140A and 1140B are disposed to cross the first gap part 1136A, the barrier 1126, and the second gap part 1136B is illustrated. As shown above, surfaces of the ultraviolet lamps 1140A and 1140B may face the surface of the first gap part 1136A, the upper surface 1126A3 of the barrier 1126, and the surface of the second gap part 1136B, respectively. In this embodiment, since the ultraviolet lamp 1140A has a cylindrical shape, the electrode connection surface 1140AB and the light extraction surface 1140AA may not be clearly distinguished from each other. However, based on FIG. 46, an upper portion may be divided into the light extraction surface 1140AA and a lower portion may be divided into the electrode connection surface 1140AB. In this state, even when the ultraviolet lamp 1140A rotates, the upper portion may still be viewed as the light extraction surface 1140AA and the lower portion may be viewed as the electrode connection surface 1140AB based on FIG. 46.

In the enlarged view of FIG. 46, a first stepped connection surface 1136A1 of the first gap part 1136A forming the first electrode part 1130A, a first stepped surface 1136A2 of the first gap part 1136A, a second stepped connection surface 1136B1 of the second gap part 1136B forming the second electrode part 1130B, and a second stepped surface 1136B2 of the second gap part 1136B are illustrated. A first electrode surface ES1 is formed in the first electrode part 1130A. The first electrode surface ES1 may include a first barrier facing surface 1135A′, the first stepped surface 1136A2, and the first stepped connection surface 1136A1. A second electrode surface ES2 is formed in the second electrode part 1130B. The second electrode surface ES12 may include a second barrier facing surface 1135B′, the second stepped surface 1136B2, and the second stepped connection surface 1136B1. In this case, a relative ratio of the separation distance (D1A) between the first stepped surface 1136A2 of the first electrode surface ES1 and the surface 1126A1 of the barrier 1126 and the separation distance (D2A) between the first stepped connection surface 1136A1 and the electrode connection surface 1141B is the same as in the above-described embodiment, and thus, a description thereof will be omitted.

It will be understood that when an element or layer is referred to as being “on” another element or layer, the element or layer can be directly on another element or layer or intervening elements or layers. In contrast, when an element is referred to as being “directly on” another element or layer, there are no intervening elements or layers present. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present invention.

Spatially relative terms, such as “lower”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “lower” relative to other elements or features would then be oriented “upper” relative to the other elements or features. Thus, the exemplary term “lower” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to cross-section illustrations that are schematic illustrations of idealized embodiments (and intermediate structures) of the disclosure. As such, variations from the shapes of the illustrations as a result, for example, of manufacturing techniques and/or tolerances, are to be expected. Thus, embodiments of the disclosure should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1. A far ultraviolet light emitting device, comprising:
a first electrode having first and second surfaces and a first electrode side surface between the first and second surfaces;
a second electrode disposed to be spaced apart from the first electrode and having first and second surfaces and a second electrode side surface between the first and second surfaces;
a barrier made of a dielectric material disposed between the first electrode side surface and the second electrode side surface; and
an ultraviolet (UV) lamp with a prescribed gas sealed in the UV lamp and having a first surface and a second surface which are opposite surfaces, the first surface of the UV lamp covering over the first surface of the first electrode, the first surface of the second electrode, and the barrier, and the second surface of the UV lamp having a light extraction surface,
wherein the prescribed gas is configured for an excimer discharge of light through the light extraction surface when the first and second electrodes are configured to receive a voltage, and
wherein at least one of the first electrode side surface or the second electrode side surface includes a barrier facing surface within a proximity of a wall surface of the barrier and stepped surface spaced apart from the wall surface, the stepped surface being formed by a step created based on a first height of the barrier facing surface being less than a second height between the first and second surfaces at the stepped surface.

2. A far ultraviolet light emitting device, comprising:
a first electrode having first and second surfaces and a first electrode side surface between first and second surfaces;
a second electrode disposed to be spaced apart from the first electrode and having first and second surfaces a second electrode side surface between first and second surfaces;
a barrier formed of a dielectric material disposed between the first electrode side surface and the second electrode side surface; and
an ultraviolet (UV) lamp with a prescribed gas sealed in the UV lamp and having a first surface and a second surface which are opposite surfaces, the first surface of the UV lamp extending over the first surface of the first electrode, the first surface of the second electrode, and the barrier, and the second surface of the UV lamp having a light extraction surface,
wherein the prescribed gas is configured for an excimer discharge of light, which is emitted through the light extraction surface, when the first and second electrodes are configure to receive a voltage,
wherein at least one of the first electrode side surface or the second electrode side surface include a recess to form a gap between the recess, the first surface of the UV lamp and the barrier.

3. An appliance or machinery having a far ultraviolet light emitting device of claim 1; and
a power supply configured to supply voltage to the first electrode and the second electrode.

4. The far ultraviolet light emitting device of claim 1, wherein at least one of the first surface or the second surface of the UV lamp is planar, and the barrier facing surface is in contact with the barrier.

5. The far ultraviolet light emitting device of claim 1, wherein the step includes a stepped connection surface connecting the barrier-facing surface and the stepped surface.

6. The far ultraviolet light emitting device of claim 1, wherein a gap is provided between the step, the wall surface of the barrier wall and the first surface of the UV lamp.

7. The far ultraviolet light emitting device of claim 1, further comprising a filter provided over the second surface of the UV lamp, the filter configured to attenuate transmission of light outside a wavelength range of light between 200 nm and 230 nm.

8. The far ultraviolet light emitting device of claim 1, wherein a first distance between the stepped surface and the barrier facing surface is 0.8 to 1.2 times a second distance corresponding to a difference between the first and second heights.

9. The far ultraviolet light emitting device of claim 8, wherein a thickness of the barrier in the direction separating the first and second electrode is less than a sum of the first and second distances.

10. The far ultraviolet light emitting device of claim 1, wherein the step of at least one of the first electrode or the second electrode creates a protrusion extending from the stepped surface to the barrier facing surface, and a groove is provided in a direction between the first and second surfaces such that the protrusion includes a first protrusion and a second protrusion spaced apart from each other.

11. The far ultraviolet light emitting device of claim 10, wherein the barrier further includes a barrier protrusion located in a space separating the first and second protrusions.

12. The far ultraviolet light emitting device of claim 1, wherein the step includes an inclined surface or a curved surface.

13. The far ultraviolet light emitting device of claim 1, further comprising a lamp housing having a mounting space, wherein the barrier forms a wall to separate the mounting space into a first mounting space configure to fit the first electrode and a second mounting space configured to fit the second electrode.

14. The far ultraviolet light emitting device of claim 13, wherein the lamp housing includes a housing body and first and second fences provided on opposite sides of the housing body and extending in a longitudinal direction of the lamp housing to form the mounting space.

15. The far ultraviolet light emitting device of claim 14, wherein a top the first and second fences include an inclined surface.

16. The far ultraviolet light emitting device of claim 13, further comprising first and second holders provided on opposite ends of the lamp housing in the longitudinal direction to hold the UV lamp, the first and second electrodes and the lamp housing together.

17. The far ultraviolet light emitting device of claim 16, wherein opposite ends of the UV lamp in contact with the first and second holders have a thickness less than a distance between the first and second surfaces of the UV lamp.

18. A far ultraviolet light emitting device, comprising:
a base;
first and second electrodes provided on the base and separated from each other, the first and second electrodes configured to have a potential difference therebetween based on voltage from an external power source;
a glass tube with a prescribed gas sealed in the gas tube, the glass tube extending over and between the first and second electrodes, the glass tube having first and second sides, which are opposite sides, wherein the first side faces the first and second electrodes, and the second side includes a light extraction surface, the glass tube configured to emit light when the external power source provides the voltage to create the potential difference exists between the first and second electrodes; and
a filter in contact with the light extraction surface, the filter configured to attenuate transmission of light outside a wavelength range of light between 190 nm to 230 nm emitted through the light extraction surface of the glass tube.

19. The far ultraviolet light emitting device of claim 18, wherein a bottom surface the first side is flat and a top surface of the second side is curved, a height of the top surface at a central axis extending from first and second electrodes being greater than a height of the top surface at opposite sides of the glass tube, where the height is based on a direction between the first and second sides.

20. The far ultraviolet light emitting device of claim 19, wherein a recessed groove extending between the first and second electrodes is provided on the bottom surface of the first side, the groove facing the first and second electrodes.
